(19) United States
(12) Patent Application Publication
(10) Pub. No.: US 2024/0390372 A1
(43) Pub. Date: Nov. 28, 2024

(54) METHODS OF TREATING HAIR-LOSS DISORDERS WITH TYK2 INHIBITORS

(71) Applicant: Bristol-Myers Squibb Company, Princeton, NJ (US)

(72) Inventors: Ian MacQuarie Catlett, Princeton, NJ (US); Jin Kim, Princeton, NJ (US); Marta Bertolini, Münster (DE); Janin Edelkamp, Münster (DE); Thomas Rouille, Münster (DE)

(21) Appl. No.: 18/694,424

(22) PCT Filed: Sep. 22, 2022
(86) PCT No.: PCT/US2022/044346

§ 371 Date: Mar. 22, 2024

Related U.S. Application Data
(60) Provisional application No. 63/247,672, filed on Sep. 23, 2021.

(51) Int. Cl. A61K 31/506; A61K 31/501; A61P 17/14

(52) U.S. Cl. CPC A61K 31/506; A61K 31/501; A61P 17/14

(57) ABSTRACT

Methods of preventing or treating an immune-mediated hair-loss disorder such as alopecia areata in a mammalian subject include administering an inhibitor of TYK2 to the mammalian subject. TYK2 inhibitors useful in such methods include a compound having the structure of Formula (I) as set forth herein, and a compound having the structure of Formula (II) as set forth herein.

### FIELD OF THE INVENTION

The present invention generally relates to methods of preventing or treating a hair-loss disorder with a tyrosine kinase 2 (TYK2) inhibitor.

### BACKGROUND

Alopecia areata (AA) is an immune-mediated disease of the hair follicle that results in non-scarring hair loss. Individuals with alopecia areata often experience chronic or relapsing disease, with severe psychological consequences. In part due to an incomplete understanding of AA pathogenesis, and despite a clinical need for therapy, AA patients have limited treatment options. The present invention addresses such need by providing novel approaches for the treatment and management of hair-loss disorders including alopecia areata.

### SUMMARY OF THE INVENTION

Described herein are methods of treating a hair-loss disorder in a subject, the methods comprising administering to the subject a TYK2 inhibitor. In some embodiments, the TYK2 inhibitor is a compound having the structure of Formula (I):

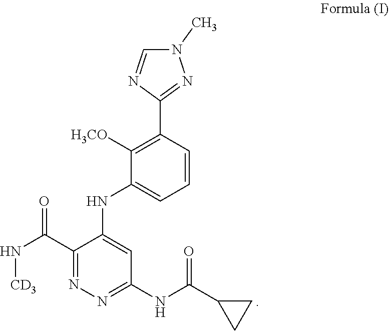 [SMILES: [2H]C([2H])([2H])NC(=O)c1nnc(NC(=O)C2CC2)cc1Nc1cccc(-c2ncn(C)n2)c1OC]

This compound is also known as deucravacitinib. In certain embodiments, the TYK2 inhibitor is a compound having the structure of Formula (II):

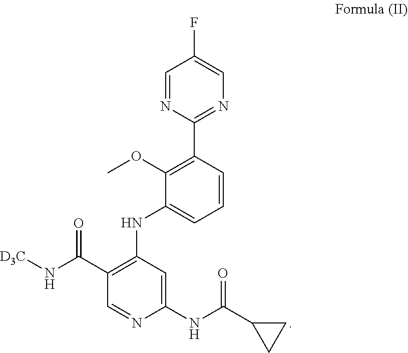 [SMILES: [2H]C([2H])([2H])NC(=O)c1cnc(NC(=O)C2CC2)cc1Nc1cccc(-c2ncc(F)cn2)c1OC]

In certain embodiments, the TYK2 inhibitor is a pharmaceutically-acceptable salt of a compound having the structure of Formula (I), or is a pharmaceutically-acceptable salt of a compound having the structure of Formula (II).

In certain embodiments, the hair-loss disorder is alopecia areata (AA). For example, in some embodiments, the methods comprise administering a TYK2 inhibitor to a subject suffering from alopecia areata. Alopecia areata includes various phenotypic subtypes such as patchy-type alopecia areata, alopecia totalis, and alopecia universalis. The TYK2 inhibitor may be administered orally, locally (such as by topical administration or local injection to the affected skin area(s)), or both orally and locally.

In some embodiments, the subject suffers from alopecia totalis or alopecia universalis. For example, certain embodiments of the invention relate to methods of treating alopecia totalis in a subject, the methods comprising administering a TYK2 inhibitor to the subject.

Described herein are also methods of preventing hair loss in a subject who has previously suffered from a hair-loss disorder (such as, e.g., alopecia areata), the methods comprising administering to the subject a TYK2 inhibitor. In some embodiments, the TYK2 inhibitor is a compound having the structure of Formula (I). In certain embodiments, the TYK2 inhibitor is a compound having the structure of Formula (II). The TYK2 inhibitor may be administered orally, locally (such as by topical administration or local injection to the previously affected skin area(s)), or both orally and locally.

### BRIEF DESCRIPTION OF THE DRAWINGS

FIG. 1 is a graph showing relative expression of MHC class I (MHC-I) in the proximal outer root sheath of hair follicles treated with vehicle, IL-12 and IL-18, or IFNγ.

FIG. 2 is a graph showing relative expression of MHC-I in the dermal cup of hair follicles treated with vehicle, IL-12 and IL-18, or IFNγ.

FIG. 3 is a graph showing the relative number of MHC class II-positive (MHC-II+) cells in the proximal outer root sheath of hair follicles treated with vehicle, IL-12 and IL-18, or IFNγ.

FIG. 4 is a graph showing the relative number of MHC-II+ cells in the bulbar connective tissue sheath of hair follicles treated with vehicle, IL-12 and IL-18, or IFNγ.

FIG. 5 is a graph showing relative expression of MICA/B in the proximal outer root sheath of hair follicles treated with vehicle, IL-12 and IL-18, or IFNγ.

FIG. 6 is a graph showing relative expression of MICA/B in the dermal cup of hair follicles treated with vehicle, IL-12 and IL-18, or IFNγ.

FIG. 7 shows images of immunostaining of hair follicles for MHC-I. The left image shows a hair follicle treated with vehicle; the middle image shows a hair follicle treated with IL-12 and IL-18; and the right image shows a hair follicle treated with IFNγ.

FIG. 8 shows images of immunostaining of hair follicles for MHC-II. The left image shows a hair follicle treated with vehicle; the middle image shows a hair follicle treated with IL-12 and IL-18; and the right image shows a hair follicle treated with IFNγ.

FIG. 9 shows images of immunostaining of hair follicles for MICA/B. The left image shows a hair follicle treated with vehicle; the middle image shows a hair follicle treated with IL-12 and IL-18; and the right image shows a hair follicle treated with IFNγ.

FIG. 10 is a graph showing the relative number of CD3+ T cells per hair follicle, as measured in the mesenchyme and in the epithelium of hair follicles treated with vehicle, IL-12 and IL-18, or IFNγ.

FIG. 11 is a graph showing the relative number of CD56+ NK cells per hair follicle, as measured in the mesenchyme and in the epithelium of hair follicles treated with vehicle, IL-12 and IL-18, or IFNγ.

FIG. 12 shows images of immunostaining of hair follicles for CD3 and CD56. The top image shows a hair follicle treated with vehicle; the middle image shows a hair follicle treated with IL-12 and IL-18; and the bottom image shows a hair follicle treated with IFNγ. Bright areas in the top and middle images are areas of CD3 or CD56 staining. Bright areas in the bottom image are areas of CD3 staining. Some of the areas of staining are indicated by arrows.

FIG. 13 shows the results of differential gene expression analysis, comparing gene expression in hair follicles treated with IL-12 and IL-18, to gene expression in hair follicles treated with vehicle.

FIG. 14 is a graph showing the number of upregulated genes in hair follicles treated with IL-12 and IL-18, compared to hair follicles treated with vehicle, when genes are grouped by particular pathways or functions. For example, for IFNγ-inducible genes, 35 out of 78 such genes were upregulated in hair follicles treated with IL-12 and IL-18, compared to hair follicles treated with vehicle; for genes associated with antigen-presenting machinery, 27 out of 123 such genes were upregulated in hair follicles treated with IL-12 and IL-18, compared to hair follicles treated with vehicle.

FIG. 15 shows the results of differential gene expression analysis, comparing gene expression in hair follicles treated with IFNγ, to gene expression in hair follicles treated with vehicle.

FIG. 16 shows the results of differential gene expression analysis, comparing gene expression in hair follicles treated with IL-12 and IL-18, to gene expression in hair follicles treated with IFNγ.

FIG. 17 is a graph showing relative expression of MHC-I in the proximal outer root sheath of hair follicles treated with vehicle, the compound of Formula (II) (which is denoted “BMS” in the figures), or tofacitinib, for 5-6 days; on day 2 of culture, either vehicle or the cytokines IL-12+IL-18 were added.

FIG. 18 is a graph showing relative expression of MHC-I in the dermal cup of hair follicles treated with vehicle, the compound of Formula (II), or tofacitinib, for 5-6 days; on day 2 of culture, either vehicle or the cytokines IL-12+IL-18 were added.

FIG. 19 is a graph showing the relative number of MHC-II+ cells in the proximal outer root sheath of hair follicles treated with vehicle, the compound of Formula (II), or tofacitinib, for 5-6 days; on day 2 of culture, either vehicle or the cytokines IL-12+IL-18 were added.

FIG. 20 is a graph showing the relative number of MHC-II+ cells in the bulbar connective tissue sheath of hair follicles treated with vehicle, the compound of Formula (II), or tofacitinib, for 5-6 days; on day 2 of culture, either vehicle or the cytokines IL-12+IL-18 were added.

FIG. 21 shows images of immunostaining of hair follicles for MHC-I. The left image shows a hair follicle treated with vehicle for 5-6 days; the middle image shows a hair follicle treated with vehicle for 5-6 days, and with IL-12 and IL-18 that were added on day 2 of culture; and the right image shows a hair follicle treated with the compound of Formula (II) for 5-6 days, and with IL-12 and IL-18 that were added on day 2 of culture.

FIG. 22 shows images of immunostaining of hair follicles for MHC-II. The left image shows a hair follicle treated with vehicle for 5-6 days; the middle image shows a hair follicle treated with vehicle for 5-6 days, and with IL-12 and IL-18 that were added on day 2 of culture; and the right image shows a hair follicle treated with the compound of Formula (II) for 5-6 days, and with IL-12 and IL-18 that were added on day 2 of culture.

FIG. 23 is a graph showing the relative number of CD3+ T cells per hair follicle, as measured in the mesenchyme of hair follicles treated as follows: with vehicle for 5-6 days; with vehicle for 5-6 days, and with IL-12 and IL-18 that were added on day 2 of culture; with the compound of Formula (II) for 5-6 days, and with IL-12 and IL-18 that were added on day 2 of culture; or with tofacitinib for 5-6 days, and with IL-12 and IL-18 that were added on day 2 of culture.

FIG. 24 is a graph showing the relative number of CD3+ T cells per hair follicle, as measured in the epithelium of hair follicles treated as follows: with vehicle for 5-6 days; with vehicle for 5-6 days, and with IL-12 and IL-18 that were added on day 2 of culture; with the compound of Formula (II) for 5-6 days, and with IL-12 and IL-18 that were added on day 2 of culture; or with tofacitinib for 5-6 days, and with IL-12 and IL-18 that were added on day 2 of culture.

FIG. 25 is a graph showing the relative number of CD56+ NK cells per hair follicle, as measured in the mesenchyme of hair follicles treated as follows: with vehicle for 5-6 days; with vehicle for 5-6 days, and with IL-12 and IL-18 that were added on day 2 of culture; with the compound of Formula (II) for 5-6 days, and with IL-12 and IL-18 that were added on day 2 of culture; or with tofacitinib for 5-6 days, and with IL-12 and IL-18 that were added on day 2 of culture.

FIG. 26 is a graph showing the relative number of CD56+ NK cells per hair follicle, as measured in the epithelium of hair follicles treated as follows: with vehicle for 5-6 days; with vehicle for 5-6 days, and with IL-12 and IL-18 that were added on day 2 of culture; with the compound of Formula (II) for 5-6 days, and with IL-12 and IL-18 that were added on day 2 of culture; or with tofacitinib for 5-6 days, and with IL-12 and IL-18 that were added on day 2 of culture.

FIG. 27 shows images of immunostaining of hair follicles for CD3 and CD56. The left image shows a hair follicle treated with vehicle for 5-6 days; the middle image shows a hair follicle treated with vehicle for 5-6 days, and with IL-12 and IL-18 that were added on day 2 of culture; and the right image shows a hair follicle treated with the compound of Formula (II) for 5-6 days, and with IL-12 and IL-18 that were added on day 2 of culture. Bright areas are areas of CD3 or CD56 staining. Some of the areas of staining are indicated by arrows.

FIG. 28 is a graph showing relative expression of MHC-I in the proximal outer root sheath of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 29 is a graph showing relative expression of MHC-I in the dermal cup of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 30 is a graph showing the relative number of MHC-II+ cells in the proximal outer root sheath of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 31 is a graph showing the relative number of MHC-II+ cells in the bulbar connective tissue sheath of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 32 shows images of immunostaining of hair follicles for MHC-I. The left image shows a hair follicle treated with vehicle for 5-6 days; the middle image shows a hair follicle treated with IL-12 and IL-18 for 5-6 days; and the right image shows a hair follicle treated with IL-12 and IL-18 for 5-6 days, and with the compound of Formula (II) that was added on day 2 of culture.

FIG. 33 shows images of immunostaining of hair follicles for MHC-II. The left image shows a hair follicle treated with vehicle for 5-6 days; the middle image shows a hair follicle treated with IL-12 and IL-18 for 5-6 days; and the right image shows a hair follicle treated with IL-12 and IL-18 for 5-6 days, and with the compound of Formula (II) that was added on day 2 of culture.

FIG. 34 is a graph showing the relative number of CD3+ T cells per hair follicle, as measured in the mesenchyme of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 35 is a graph showing the relative number of CD3+ T cells per hair follicle, as measured in the epithelium of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 36 is a graph showing the relative number of CD56+ NK cells per hair follicle, as measured in the mesenchyme of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 37 is a graph showing the relative number of CD56+ NK cells per hair follicle, as measured in the epithelium of hair follicles treated as follows: with vehicle for 5-6 days; with IL-12 and IL-18 for 5-6 days; with IL-12 and IL-18 for 5-6 days, and with the compound of Formula (II) that was added on day 2 of culture; or with IL-12 and IL-18 for 5-6 days, and with tofacitinib that was added on day 2 of culture.

FIG. 38 shows images of immunostaining of hair follicles for CD3 and CD56. The left image shows a hair follicle treated with vehicle for 5-6 days; the middle image shows a hair follicle treated with IL-12 and IL-18 for 5-6 days; and the right image shows a hair follicle treated with IL-12 and IL-18 for 5-6 days, and with the compound of Formula (II) that was added on day 2 of culture. Bright areas are areas of CD3 or CD56 staining. Some of the areas of staining are indicated by arrows.

FIG. 39 is a graph showing relative production of IFNγ from hair follicles treated as follows: with vehicle; pre-treated with vehicle, with IL-12 and IL-18 later added to the culture medium; pre-treated with the compound of Formula (II), with IL-12 and IL-18 later added to the culture medium; or pre-treated with tofacitinib, with IL-12 and IL-18 later added to the culture medium.

FIG. 40 is a graph showing relative production of IFNγ from hair follicles treated as follows: with vehicle; with IL-12 and IL-18; pre-treated with IL-12 and IL-18, with the compound of Formula (II) later added to the culture medium; pre-treated with IL-12 and IL-18, with tofacitinib later added to the culture medium.

FIG. 41 is a graph showing the relative number of IL-12RB2 positive cells in hair follicles obtained from healthy donors, patients with acute AA, and patients with chronic AA.

FIG. 42 shows images of immunostaining of hair follicles for IL-12RB2.

### DETAILED DESCRIPTION

The present disclosure relates in part to the discovery that local IL-12 is important in AA pathogenesis. As shown in the Examples, IL-12, supported by IL-18, is sufficient to induce IFNγ secretion and hair follicle immune privilege collapse. In addition, IL-12 receptor positive immune cells are present around affected hair bulbs in AA patients. Based on these findings, the present disclosure further relates to the discovery that inhibition of tyrosine kinase 2 (TYK2) can prevent IL-12-induced hair follicle immune privilege collapse. TYK2 inhibition can also restore immune privilege of the hair follicle, after immune privilege collapse has been induced by IL-12 and IL-18. The ability to restore immune privilege of the hair follicle, via inhibition of TYK2, demonstrates that TYK2 inhibition in AA patients is a viable therapeutic strategy.

TYK2 is a member of the Janus kinase (JAK) family of nonreceptor tyrosine kinases and has been shown to be critical in regulating the signal transduction cascade downstream of receptors for IL-12, IL-23, and type I interferons in both mice (Ishizaki, M. et al., “Involvement of tyrosine kinase-2 in both the IL-12/Th1 and IL-23/Th17 axes in vivo,” J. Immunol., 187:181-189 (2011); Prchal-Murphy, M. et al., “TYK2 kinase activity is required for functional type I interferon responses in vivo,” PLOS One, 7: e39141 (2012)) and humans (Minegishi, Y. et al., “Human tyrosine kinase 2 deficiency reveals its requisite roles in multiple cytokine signals involved in innate and acquired immunity,” Immunity, 25:745-755 (2006)). TYK2 mediates the receptor-induced phosphorylation of members of the STAT family of transcription factors, an essential signal that leads to the dimerization of STAT proteins and the transcription of STAT-dependent pro-inflammatory genes. TYK2-deficient mice are resistant to experimental models of colitis, psoriasis, and multiple sclerosis, demonstrating the importance of TYK2-mediated signaling in autoimmunity and related disorders (Ishizaki, M. et al., “Involvement of tyrosine kinase-2 in both the IL-12/Th1 and IL-23/Th17 axes in vivo,” J. Immunol., 187:181-189 (2011); Oyamada, A. et al., “Tyrosine kinase 2 plays critical roles in the pathogenic CD4 T cell responses for the development of experimental autoimmune encephalomyelitis,” J. Immunol., 183:7539-7546 (2009)).

In humans, individuals expressing an inactive variant of TYK2 are protected from multiple sclerosis and possibly other autoimmune disorders (Couturier, N. et al., “Tyrosine kinase 2 variant influences T lymphocyte polarization and multiple sclerosis susceptibility,” Brain, 134:693-703 (2011)). Genome-wide association studies have shown other variants of TYK2 to be associated with autoimmune disorders such as Crohn's disease, psoriasis, systemic lupus erythematosus, and rheumatoid arthritis, further demonstrating the importance of TYK2 in autoimmunity (Ellinghaus, D). et al., “Combined Analysis of Genome-wide Association Studies for Crohn Disease and Psoriasis Identifies Seven Shared Susceptibility Loci,” Am. J. Hum. Genet., 90:636-647 (2012); Graham, D. et al., “Association of polymorphisms across the tyrosine kinase gene, TYK2 in UK SLE families,” Rheumatology (Oxford), 46:927-930 (2007); Eyre, S. et al., “High-density genetic mapping identifies new susceptibility loci for rheumatoid arthritis,” Nat. Genet., 44:1336-1340 (2012)).

The present disclosure generally relates to using inhibitors to TYK2 to treat immune-mediated hair-loss disorders, such as alopecia areata. Alopecia areata is a disease characterized by lesional hair follicles infiltrated by inflammatory T cells and NK cells. The healthy hair follicle epithelium is a site of immune privilege, such that the hair follicle is protected from auto-inflammatory immune responses; such immune privilege is due to the relative immunosuppressive environment achieved by, e.g., the downregulation or absence of MHC class I and MHC class II expression. In contrast to healthy hair follicles, hair follicles in AA have higher expression of MHC-I and MHC-II. Such immune privilege collapse of the hair follicle in AA is believed to be the cause of hair loss. Bertolini et al., “Hair follicle immune privilege and its collapse in alopecia areata,” Experimental Dermatology, 29:1-23 (2020). While IFNγ is regarded as a key cytokine driving AA, the early stages and events of disease onset are not yet fully understood.

The Examples provided herein show that TYK2 inhibition can prevent immune privilege collapse and moreover can restore immune privilege of the hair follicle. As shown herein, local IL-12 signaling may be critical during the early stages and maintenance of AA pathogenesis by promoting IFNγ production from resident IL-12RB2+ immune cells, leading to hair follicle immune privilege collapse. The inhibition of TYK2-dependent, IL-12-mediated signaling prevents hair follicle immune privilege collapse, and treatment with a selective TYK2 inhibitor can restore immune privilege (after collapse), as demonstrated herein. These findings support targeting TYK2 for pharmacological AA therapy.

TYK2 inhibitors useful for the methods described herein include compounds disclosed in U.S. Pat. No. RE47,929 E, the contents of which are hereby incorporated by reference herein in their entirety. For example, in certain embodiments, the TYK2 inhibitor is deucravacitinib. Deucravacitinib is also known as 6-(cyclopropanecarboxamido)-4-((2-methoxy-3-(1-methyl-1H-1,2,4-triazol-3-yl)phenyl)amino)-N-(methyl-d₃)pyridazine-3-carboxamide, having the structure of Formula (I):

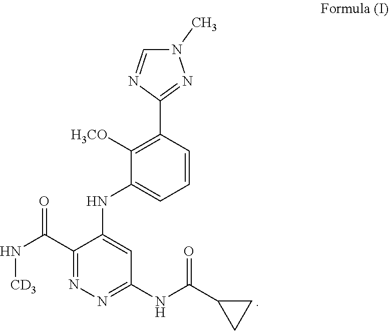 [SMILES: [2H]C([2H])([2H])NC(=O)c1nnc(NC(=O)C2CC2)cc1Nc1cccc(-c2ncn(C)n2)c1OC]

Deucravacitinib allosterically inhibits TYK2 by binding to the regulatory domain of TYK2, rather than to the enzyme's catalytic domain. Deucravacitinib is highly selective for TYK2.

Other TYK2 inhibitors that can be used in the methods described herein include compounds disclosed in U.S. Pat. No. 9,663,467, the contents of which are hereby incorporated by reference herein in their entirety. For example, in certain embodiments, the TYK2 inhibitor used in the methods described herein is a compound having the structure of Formula (II):

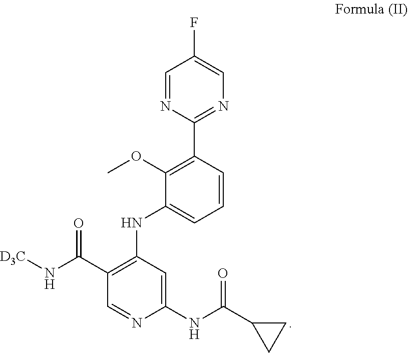 [SMILES: [2H]C([2H])([2H])NC(=O)c1cnc(NC(=O)C2CC2)cc1Nc1cccc(-c2ncc(F)cn2)c1OC]

A TYK2 inhibitor may be administered or formulated as pharmaceutically-acceptable salt, such as, e.g., a pharmaceutically-acceptable salt of the compound having the structure of Formula (I), or a pharmaceutically-acceptable salt of the compound having the structure of Formula (II). For example, the TYK2 inhibitor may be formulated as a hydrochloride salt, a methanesulfonic acid salt, or a sulfate salt, of the compound having the structure of Formula (I). See, for example, International Application Nos. PCT/US2019/034534 and PCT/US2020/036727 (published as WO 2019/232138 and WO 2020/251911, respectively), the entire contents of each of which are hereby incorporated by reference herein.

Administering a TYK2 inhibitor to treat a hair-loss disorder as described herein may comprise administering a TYK2 inhibitor systemically (e.g., orally), or locally to the affected site(s) of the skin (e.g., by topical administration or by local injection). In some embodiments, a TYK2 inhibitor is administered orally, topically, or both orally and topically. Oral dosage forms include, for example, dosage forms as described in International Application No. PCT/US2020/051342 (published as WO 2021/055652), the contents of which are hereby incorporated by reference herein in their entirety. Topical dosage forms include gels, creams, ointments, foams, and solutions, for example. Administration of the TYK2 inhibitor may comprise once daily, twice daily, or thrice daily administration. In addition, the TYK2 inhibitor may be administered over the course of several weeks (e.g., for at least two weeks) or months (e.g., for one month, two months, three months, or longer).

For any of the embodiments described herein, the dose of the TYK2 inhibitor that may be administered (e.g., orally) to a subject can range from about 1 mg to about 100 mg per day, or about 1 mg to about 40 mg per day. For example, in some embodiments, a dose of 3 mg, 6 mg, 12 mg, 15 mg, or 36 mg of the TYK2 inhibitor per day is administered to a subject in a method as described herein. Such per day doses may be administered once daily, or may be administered in two or more divided doses (for example, for a total daily dose of 12 mg, the 12 mg may be administered once daily, or may be administered as two 6 mg doses, or may be administered as three 4 mg doses). In certain embodiments, the TYK2 inhibitor is deucravacitinib. In other embodiments, the TYK2 inhibitor is a compound having the structure of Formula (II).

Certain embodiments of the present invention relate to methods of preventing a relapse of hair loss. For example, in some embodiments, a method comprises administering a TYK2 inhibitor to a subject who has previously suffered from a hair-loss disorder or who has been diagnosed with a hair-loss disorder (e.g., alopecia areata). In certain such embodiments, the subject may not currently be experiencing hair loss but has previously suffered from hair loss. In further embodiments, the subject is topically administered a TYK2 inhibitor to, for example, certain areas of the scalp.

In addition, embodiments of the invention relate to methods of treating hair loss in a subject. For example, in some embodiments, a method comprises administering a TYK2 inhibitor to a subject suffering from hair loss. Such subject may be a patient experiencing hair loss associated with alopecia areata. Administering a TYK2 inhibitor to such a subject may promote hair regrowth in the affected sites.

There are several forms of the autoimmune disease known as alopecia areata. A subject with alopecia areata may suffer from patchy hair loss on places of the body that normally grow hair (e.g., the scalp). In some cases, hair loss progresses to the entire scalp (which is known as alopecia totalis) or to the entire body (which is known as alopecia universalis). All types of alopecia areata fall within the scope of the embodiments described herein.

In any of the embodiments described herein, a subject may be administered a TYK2 inhibitor in combination with one or more other agents.

In the context of the present invention, subjects, and in particular human subjects, also may be referred to as patients.

### EXAMPLES

The invention will be further described by the following examples. The examples serve only to illustrate the invention and its practice. The examples are not to be construed as limitations on the scope or spirit of the invention.

In the following examples and the accompanying drawings, the following abbreviations apply: AA=alopecia areata; HF=hair follicle; IFNγ=interferon gamma; IL=interleukin; IR=ImmunoReactivity; MHC=major histocompatibility complex; NK=natural killer; ORS=outer root sheath; DC=dermal cup; CTS=connective tissue sheath; Tofa=tofacitinib; FC=fold change. The compound having the structure of Formula (II) is denoted as “BMS” in the accompanying drawings.

To address the role of IL-12 in AA pathobiology and its potential as a therapeutic target, microdissected hair follicles from human scalp were cultured in the presence of IL-12 (3 ng/mL) and IL-18 (20 ng/mL) and in the presence or absence of a selective allosteric TYK2 inhibitor, the compound of Formula (II) (300 nM). IFNγ (75 UI/mL) was used as a positive control to induce immune privilege (IP) collapse, and tofacitinib was used as a positive control for blocking IFNγ signaling. Hair follicle immune privilege (HF-IP) was assessed by quantitative immunohistomorphometry of MHC class I, of MHC class II, and of MHC class chain-related protein A and MHC class chain-related protein B (MICA/B).

Resident immune cell populations were assessed by quantification of CD3-positive cells and of CD56-positive cells. Gene expression of treated hair follicles was assessed using whole transcriptome analysis. IFNγ production was quantified by enzyme-linked immunosorbent assay (R&D systems). IL-12RB2-expressing cells were evaluated in healthy donors, acute AA patients, and chronic AA patients using immunohistomorphometry.

### Example 1: Assessing the Role of IL-12 in AA Pathogenesis and the Potential of IL-12 as a Therapeutic Target in AA

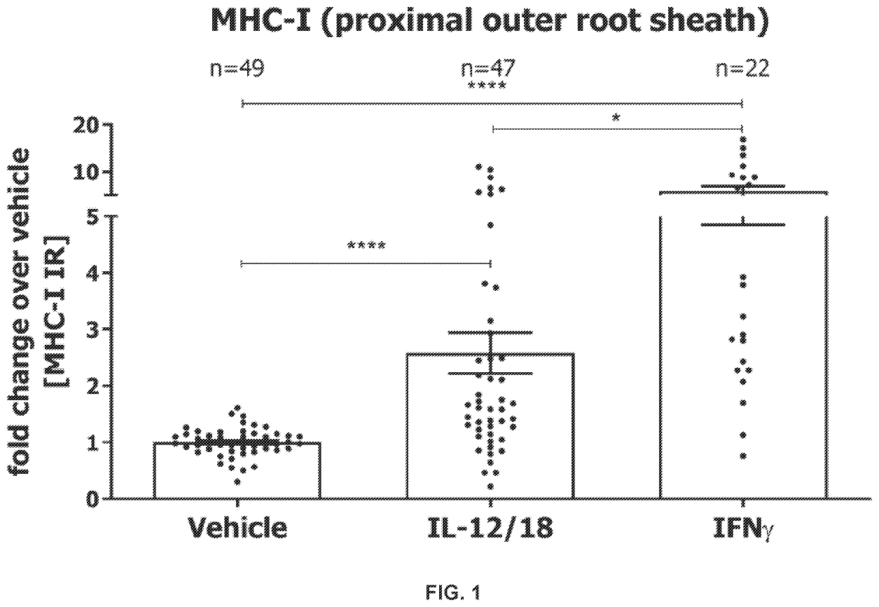

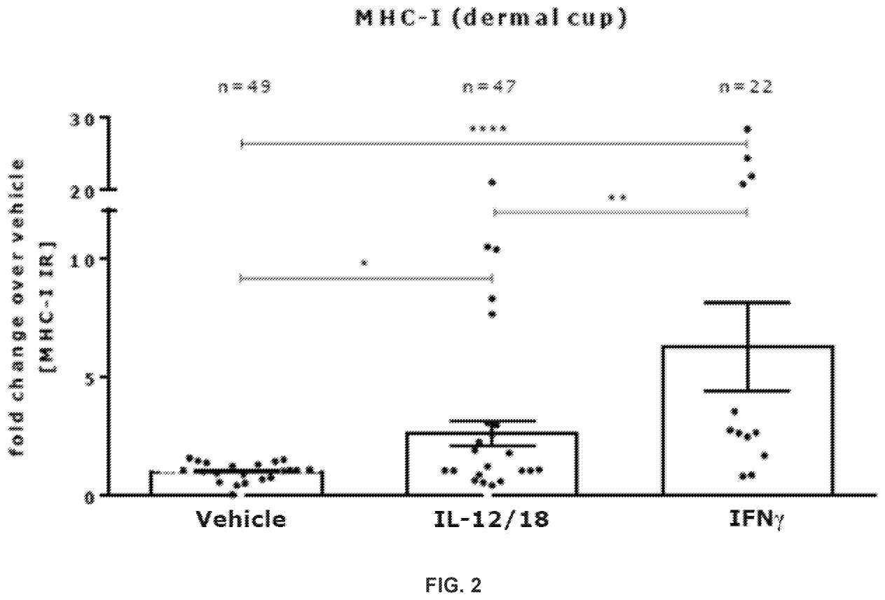

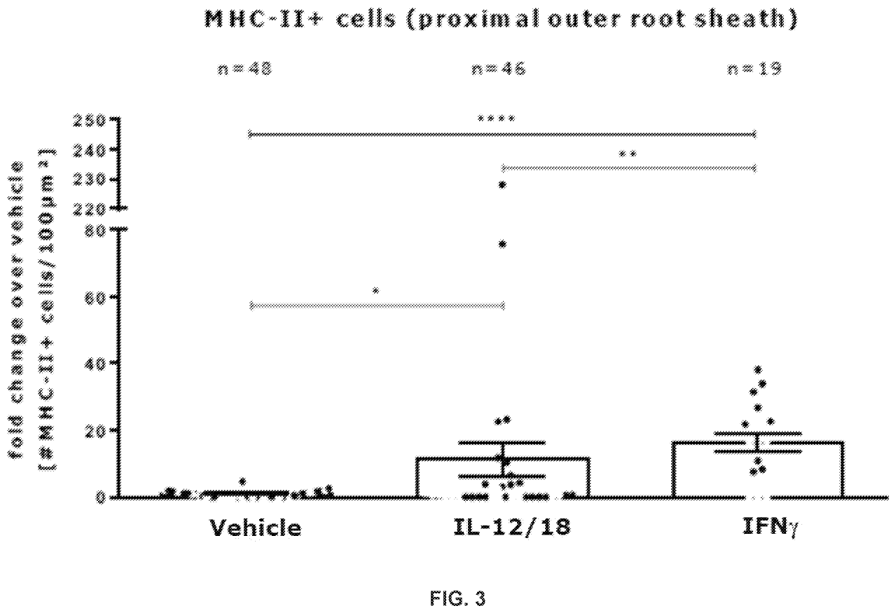

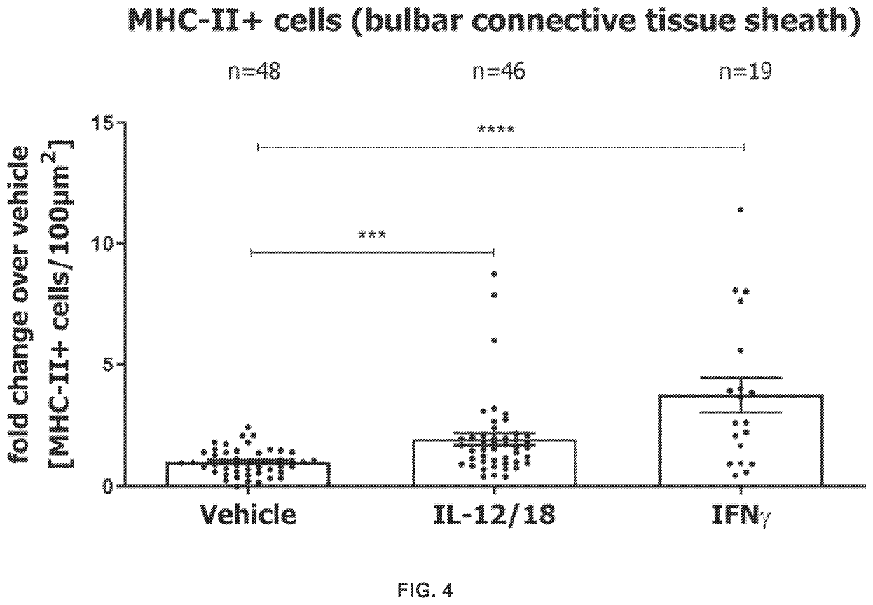

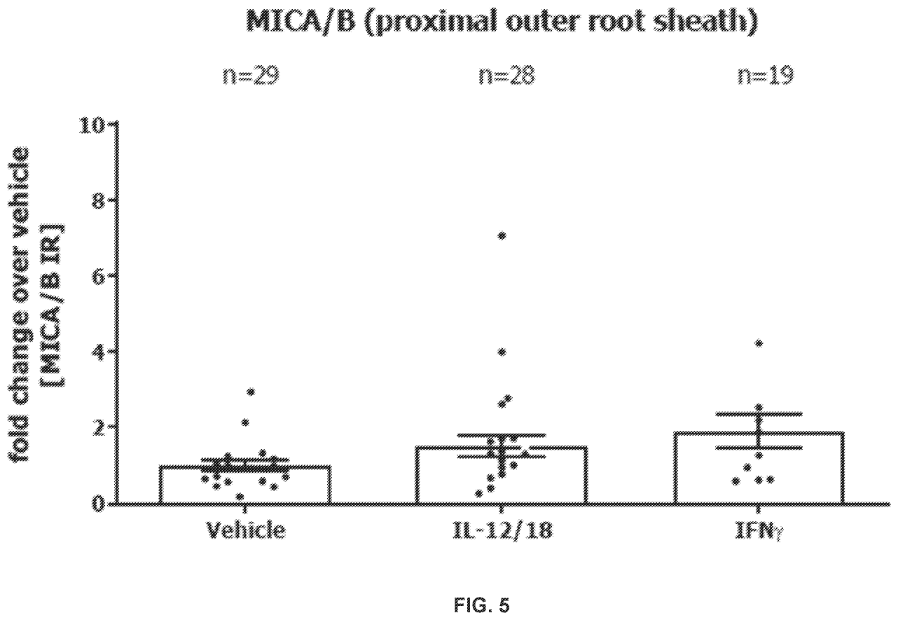

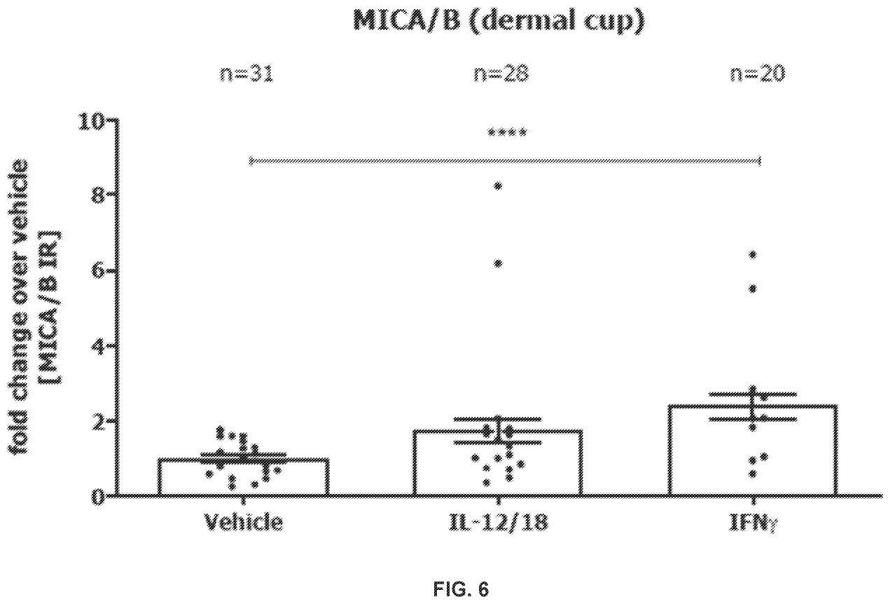

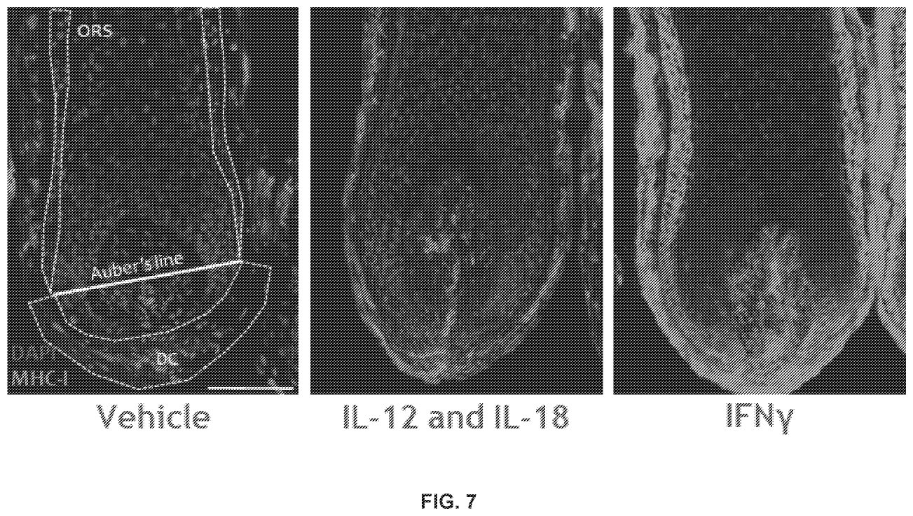

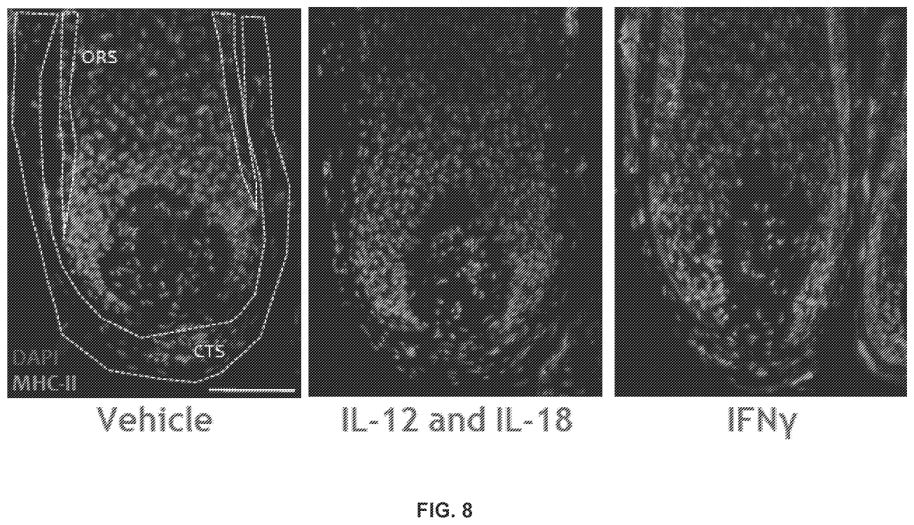

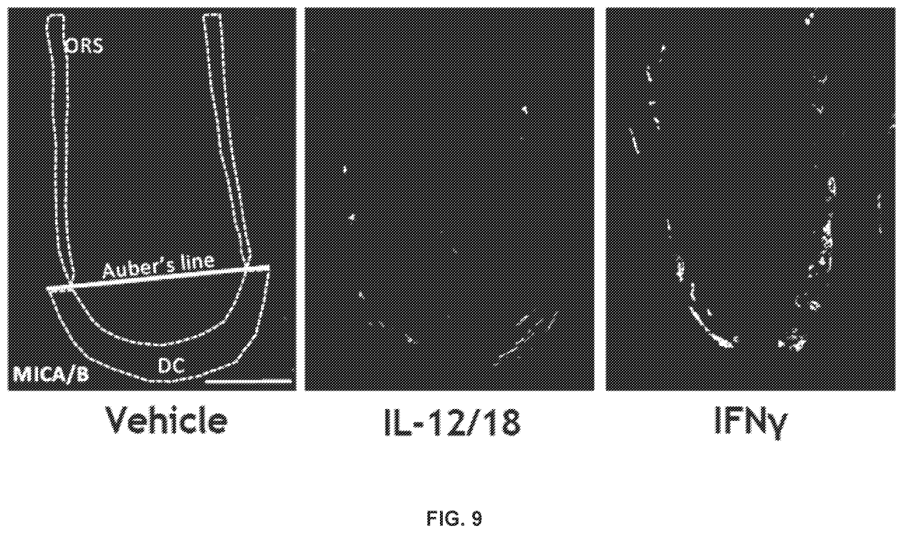

An ex vivo model of hair follicle immune privilege collapse was established. Microdissected hair follicles from human scalp were cultured ex vivo for 5-6 days in the presence of vehicle; IL-12 (3 ng/mL) and IL-18 (20 ng/ml); or IFNγ (75 UI/mL). Each group included 19-48 hair follicles, obtained from 4-9 independent healthy donors. Hair follicle immune privilege collapse was assessed by quantitative immunohistomorphometry of MHC class I and of MHC class II, and of MICA/B. See FIGS. 1-9. The graphs in FIGS. 1-6 show mean #SEM (standard error of the mean) and results of the Dunn's multiple comparison test (*p<0.05; **p<0.01; ***p<0.001; ****p<0.0001). FIGS. 7, 8, and 9 show immunostaining of MHC-I, MHC-II, and MICA/B, respectively (scale bar=100 μm).

Treatment with IL-12 and IL-18 led to a significant increase of antigen presenting molecules. Treatment with IL-12 and IL-18, compared to vehicle, increased MHC class I expression in the hair follicle outer root sheath, and similarly in the dermal cup. Treatment with IL-12 and IL-18, compared to vehicle, also increased the number of MHC class II-expressing cells in the bulbar connective tissue sheath, and increased MHC class II ectopic expression in the outer root sheath. Such MHC class II ectopic expression is a cardinal feature of HF-IP collapse.

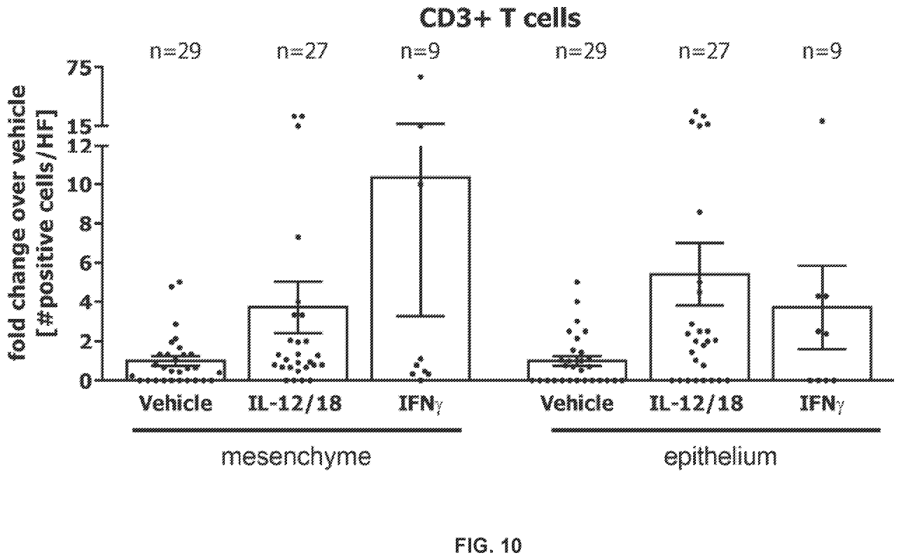

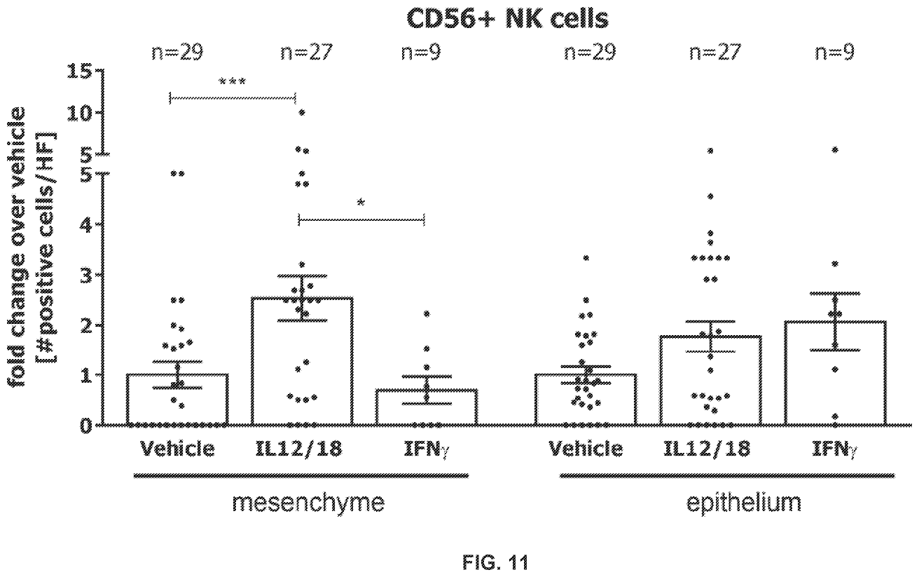

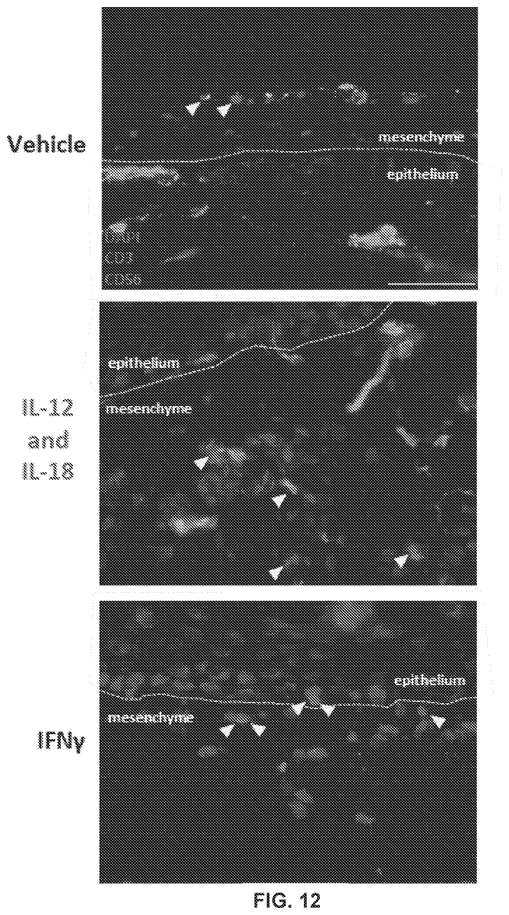

The effect of IL-12 and IL-18 treatment on T cell and NK cell expansion was also examined. Microdissected hair follicles were again treated with vehicle, with IL-12 (3 ng/mL) and IL-18 (20 ng/mL), or with IFNγ (75 UI/mL) for 5-6 days. Each group included 9-29 hair follicles, obtained from 2-6 independent healthy donors. Resident immune cell populations were assessed by quantification of CD3-positive cells and of CD56-positive cells. See FIGS. 10-12. The graphs in FIGS. 10 and 11 show mean±SEM and results of the Dunn's multiple comparison test (*p<0.05; ***p<0.001). FIG. 12 shows immunostaining of CD3 (T cells) and of CD56 (NK cells) in hair follicles (scale bar=50 μm).

IL-12+IL-18 treatment of hair follicles resulted in increased numbers of CD3+ T cells and of CD56+ NK cells in hair follicle epithelium and mesenchyme. Small numbers of T and NK cells were observed in vehicle-treated hair follicles. However, treating hair follicles with IL-12+IL-18 increased the numbers of T and NK cells. T cells and NK cells are key effector cells in AA, and these results show that treatment with IL-12 and IL-18 increases the number of these cells in healthy scalp hair follicles.

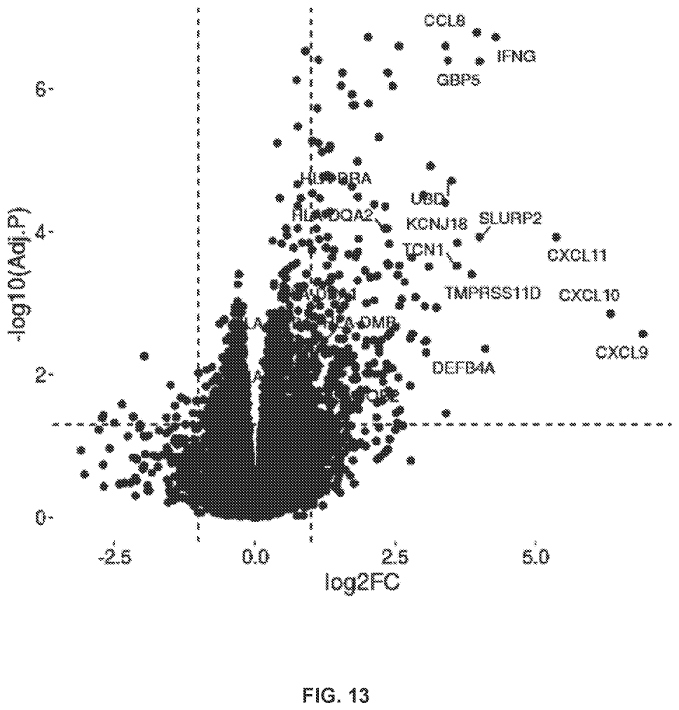

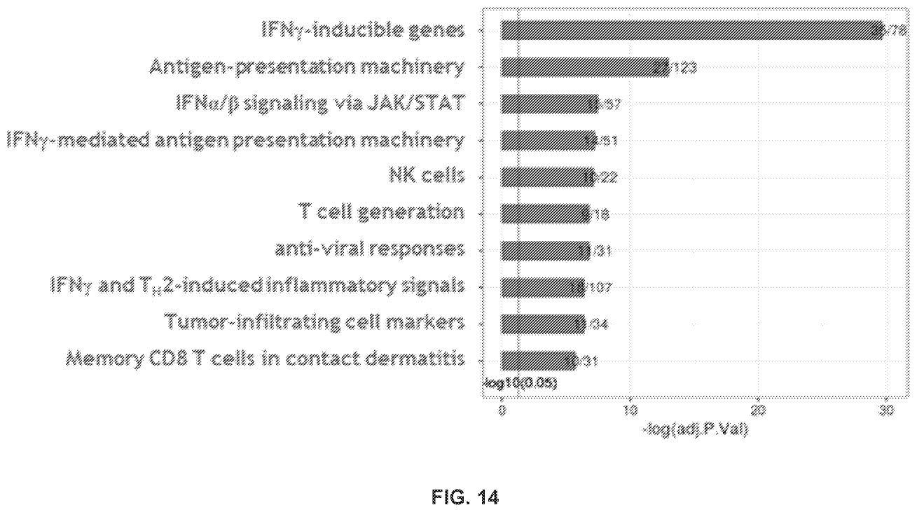

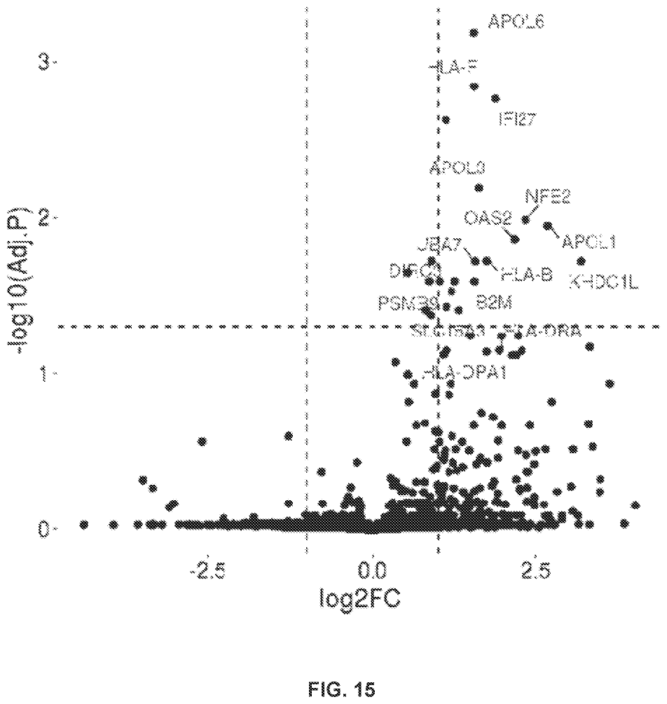

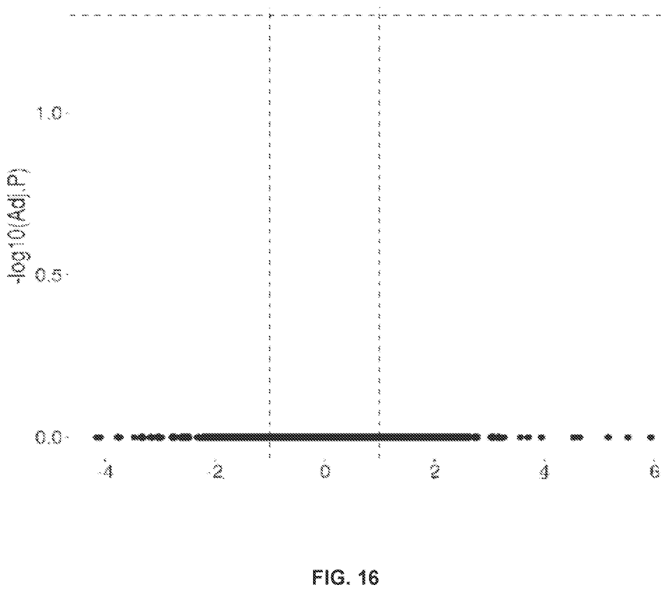

Gene expression of treated hair follicles was assessed using whole transcriptome analysis. For this analysis, 6-15 hair follicles, obtained from 2-3 independent healthy donors, were cultured with vehicle, with IL-12 (3, 4.5, or 6 ng/ml) and IL-18 (20, 30, or 40 ng/ml), or with IFNγ (75 IU/mL), for 24 hours prior to whole transcriptome analysis. FIG. 13 shows differentially expressed genes in IL-12+IL-18-treated hair follicles (from 3 donors) normalized to vehicle-treated hair follicles (from 3 donors) (dashed horizontal line indicates adjusted p<0.05). FIG. 14 shows the top ten significantly enriched pathways (vertical line in FIG. 14 indicates adjusted p<0.05) in reference to the MetaCore database. FIG. 15 shows differentially expressed genes in IFNγ-treated hair follicles (from 2 donors) normalized to vehicle-treated hair follicles (dashed horizontal line indicates adjusted p<0.05). FIG. 16 shows differentially expressed genes of IL-12+IL-18-treated hair follicles versus IFNγ-treated hair follicles (dashed horizontal line indicates adjusted p<0.05).

The above whole transcriptome and pathway analyses revealed that IL-12+IL-18 treatment of hair follicles selectively induced expression of IFNγ and of IFNγ-inducible genes, and of genes related to antigen-presentation pathways (e.g., MHC-II, consistent with the protein expression results above), as well as of chemoattractants relevant to AA (e.g., CXCL-10). Differentially regulated genes detected in IFNγ-treated hair follicles versus vehicle-treated hair follicles were similar. No significant gene expression differences were observed between IL-12+IL-18-treated hair follicles versus IFNγ-treated hair follicles (see FIG. 16). These results indicate that IL-12, supported by IL-18, induces IFNγ expression.

### Example 2: Inhibition of TYK2 Prevented IL-12+IL-18-Induced Immune Privilege Collapse of Hair Follicles

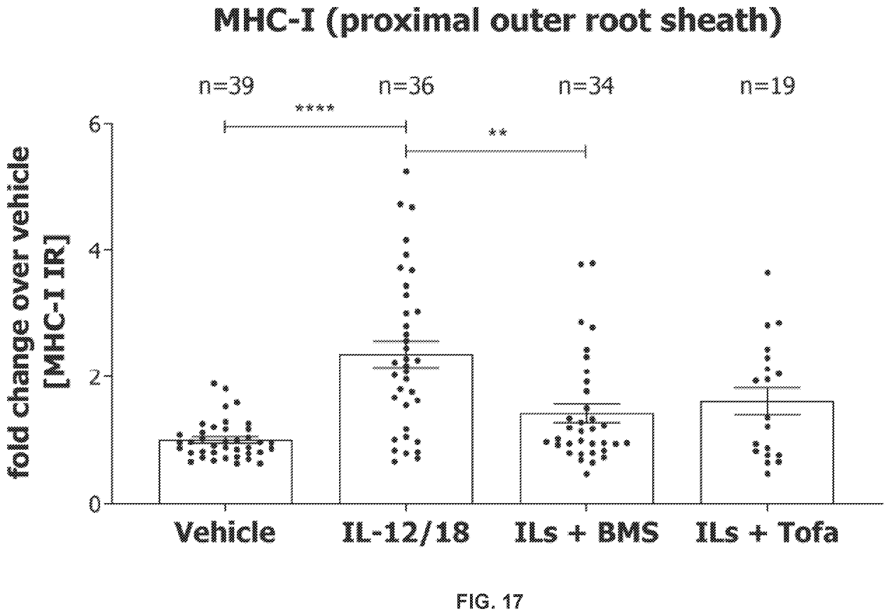

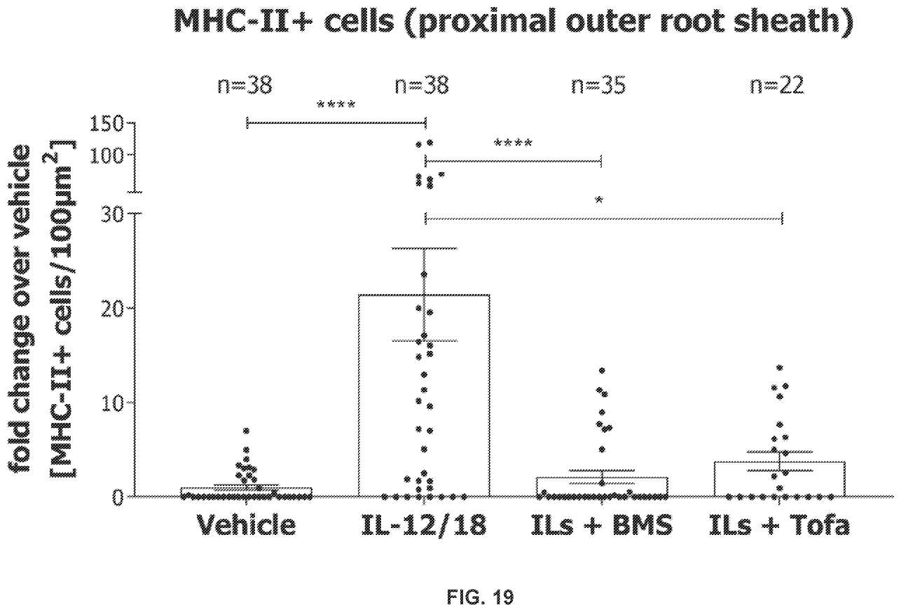

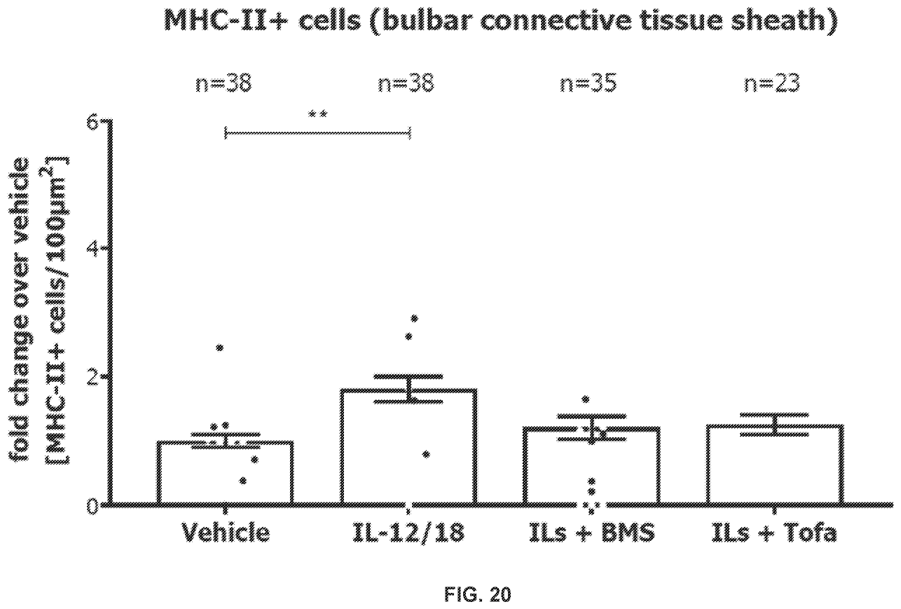

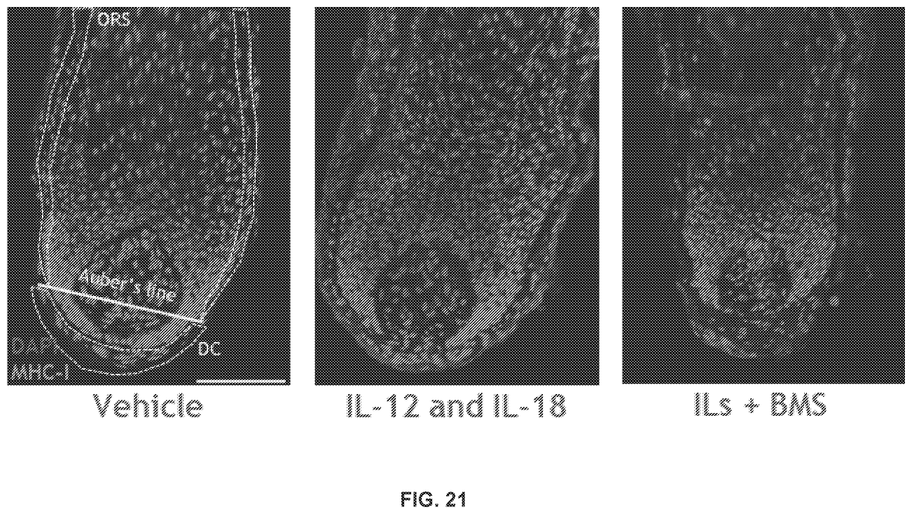

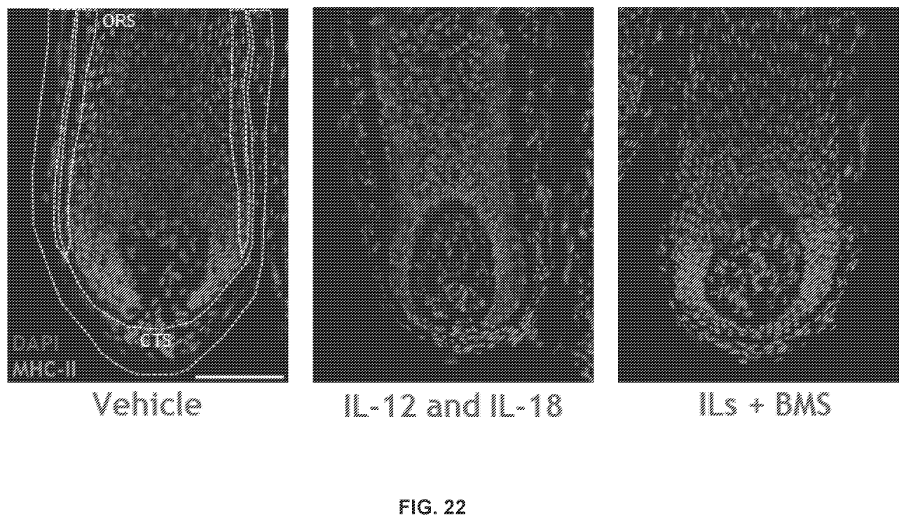

Hair follicles were pre-treated with the compound having the structure of Formula (II) (denoted “BMS” in the figures), followed by the addition of IL-12 and IL-18, in a prophylactic assay. Tofacitinib was used as a control, for comparison. For this assay, each group included 19-40 hair follicles, obtained from 3-5 independent healthy donors. The groups were treated as follows: with vehicle, with the compound of Formula (II) (300 nM), or with tofacitinib (400 nM) for 5-6 days; on day 2 of culture, vehicle, or IL-12 (3 ng/ml) and IL-18 (20 ng/mL) were added. Levels of MHC class I and of MHC class II were assessed as described above, and FIGS. 17-22 provide the results. The graphs in FIGS. 17-20 show mean±SEM and results of the Dunn's multiple comparison test (*p<0.05; **p<0.01; ****p<0.0001). FIGS. 21 and 22 show staining of MHC-I and MHC-II, respectively, in hair follicles (scale bar=100 μm).

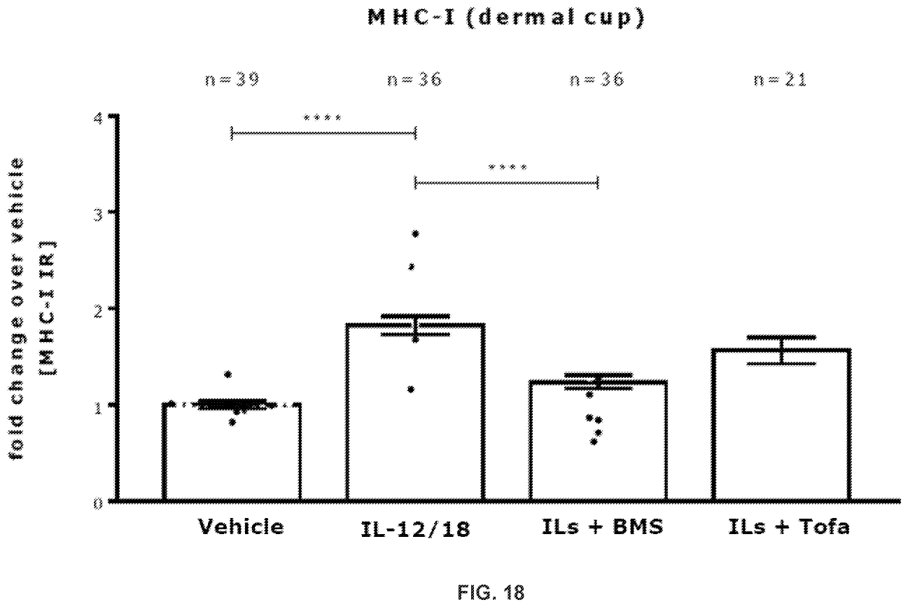

Inhibition of TYK2 with the compound of Formula (II) prevented IL-12+IL-18-mediated upregulation of MHC class I and II in the outer root sheath. The effect of TYK2 inhibition by the compound of Formula (II) was greater than the effect observed with tofacitinib. This effect was also observed in other hair follicle compartments. See, e.g., FIG. 18. These results show that pre-treatment with the compound of Formula (II) prevented IL-12 and IL-18-induced immune privilege collapse of hair follicles. These results also confirm that IL-12 signaling is required for the induction of IL-12+IL-18-mediated immune privilege collapse.

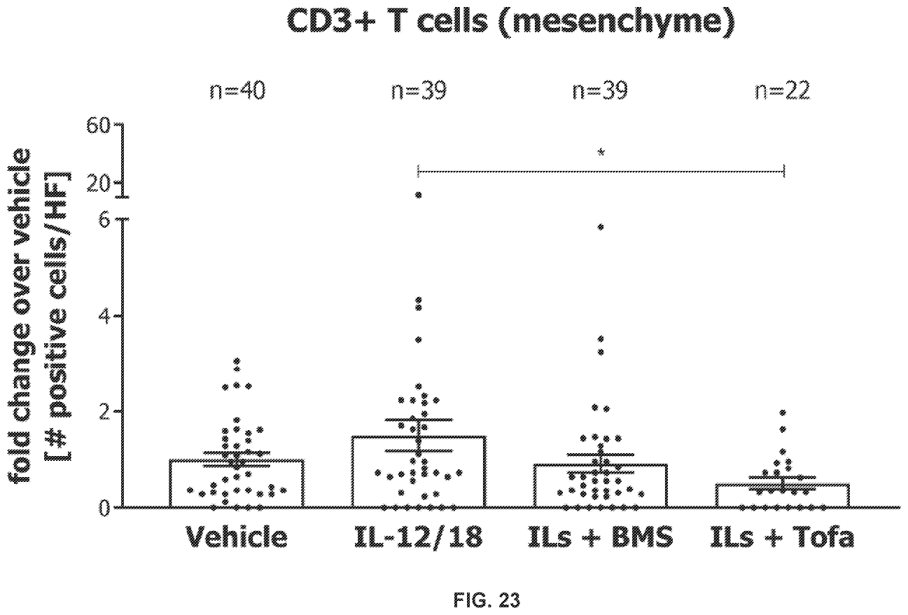

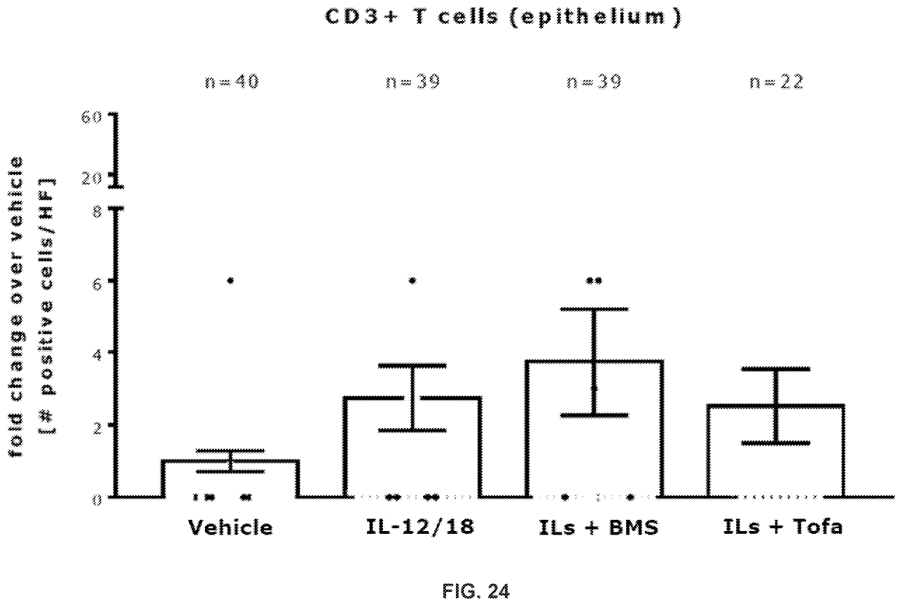

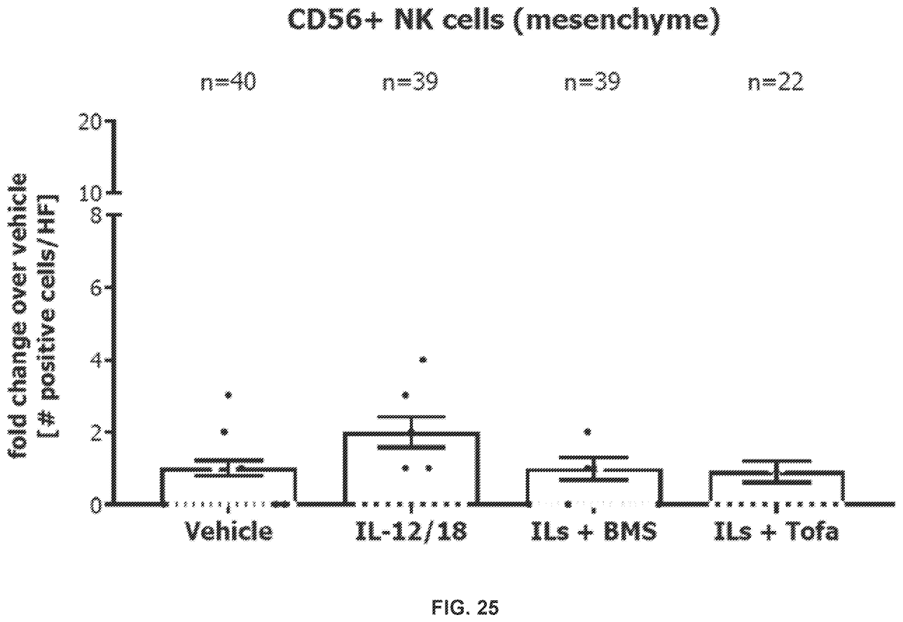

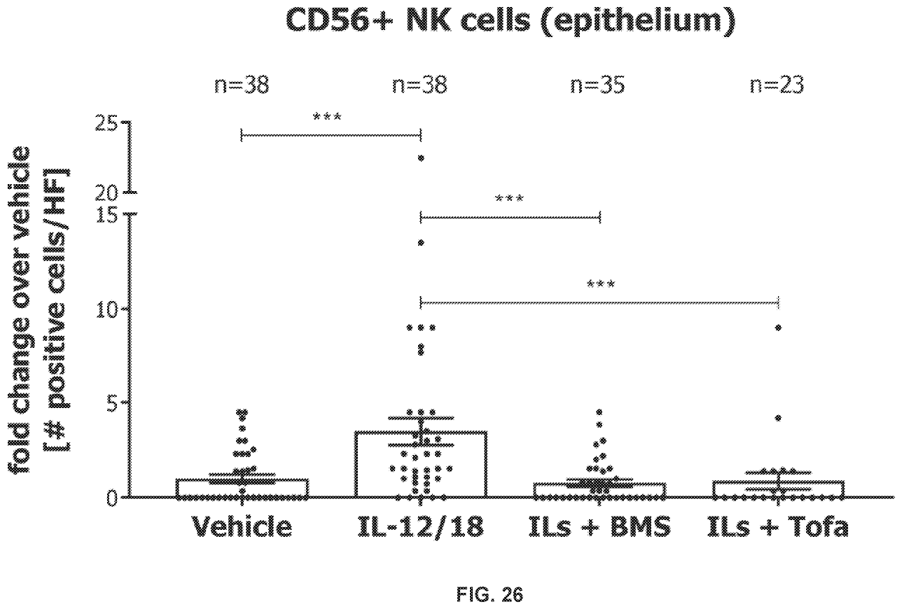

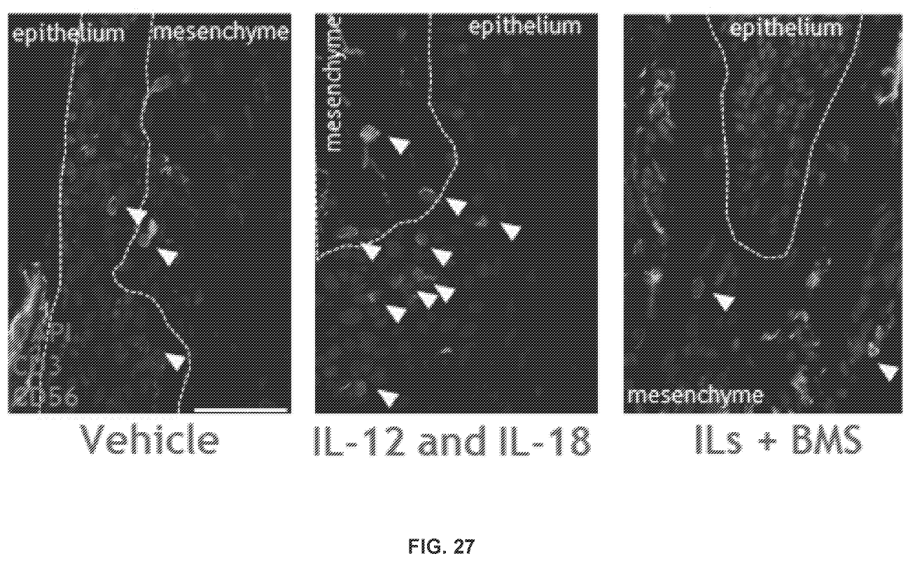

The effect of pre-treatment with the compound of Formula (II) on T cell and NK cell expansion following IL-12 and IL-18 stimulation was also examined. 22-40 hair follicles, obtained from 4-5 independent healthy donors, were incubated with vehicle, with the compound of Formula (II) (300 nM), or with tofacitinib (400 nM) for 5-6 days. On day 2 of culture, vehicle, or IL-12 (3 ng/ml) and IL-18 (20 ng/mL) were added. Resident immune cell populations were assessed by quantification of CD3-positive cells and of CD56-positive cells. FIGS. 23-27 provide the results. The graphs in FIGS. 23-26 show mean±SEM and results of the Dunn's multiple comparison test (*p<0.05; ***p<0.001). FIG. 27 shows staining of CD3 and of CD56 in hair follicles (scale bar=50 μm). Pre-treatment with the compound of Formula (II) prevented an elevation of CD3+ T cell frequency and of CD56+ NK cell frequency, consistent with the results above.

### Example 3: Inhibition of TYK2 with the Compound of Formula (II) Restored Hair Follicle Immune Privilege Following IL-12 and IL-18-Induced Immune Privilege Collapse

The above assays were adapted to investigate whether TYK2 inhibition can inhibit ongoing IL-12+IL-18-mediated hair follicle immune privilege collapse, and thus whether TYK2 inhibition is a viable therapeutic strategy for treating alopecia areata. In these assays, immune privilege collapse was first induced by treatment with IL-12+IL-18, followed by the addition of the compound of Formula (II).

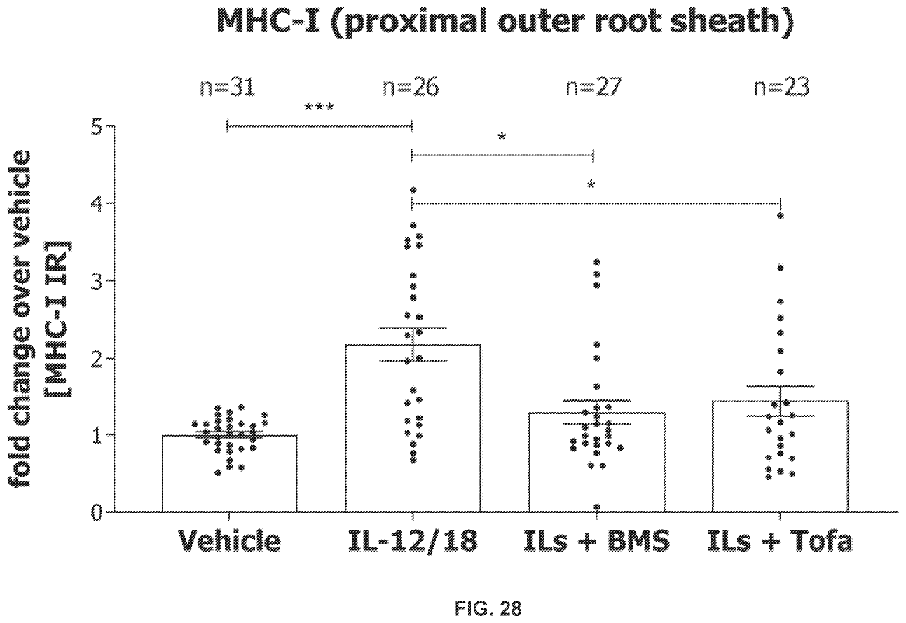

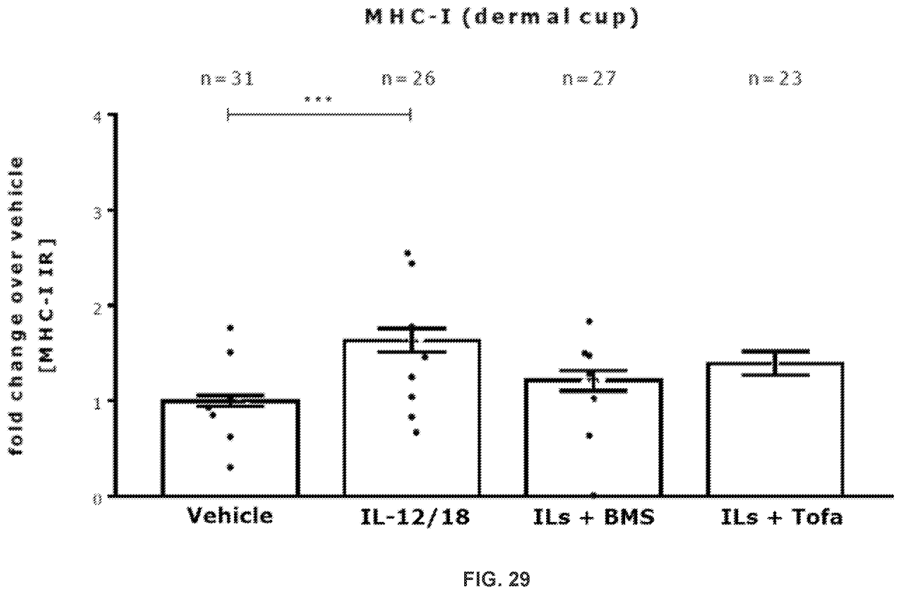

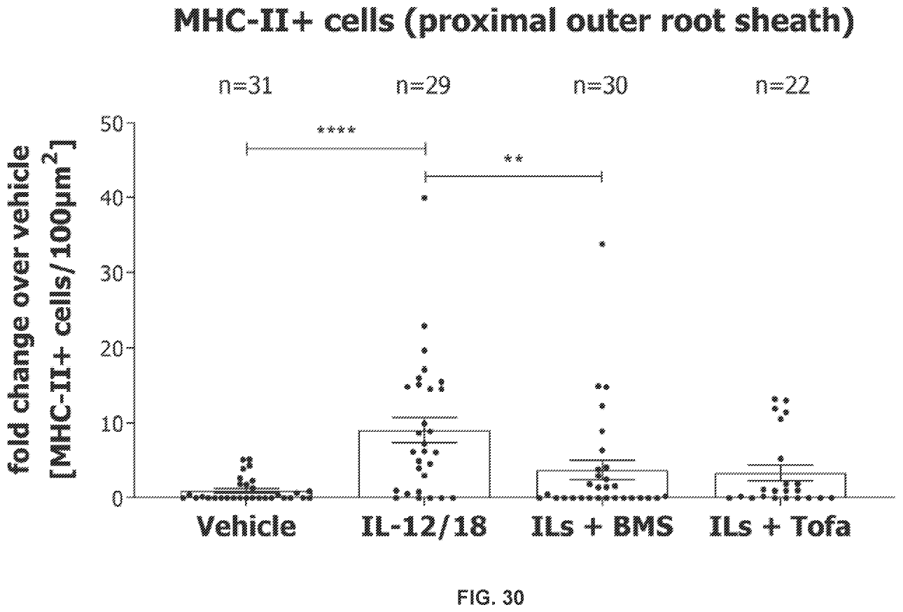

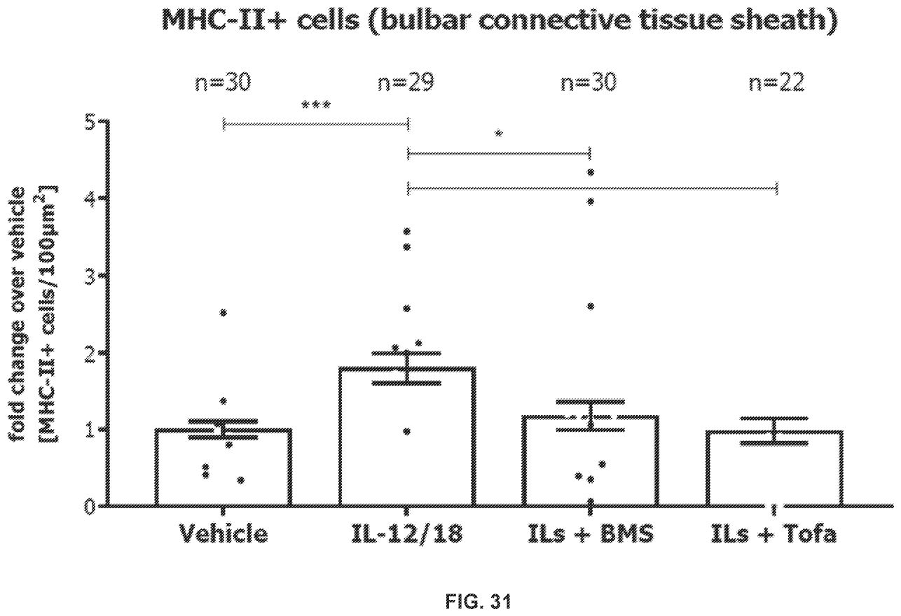

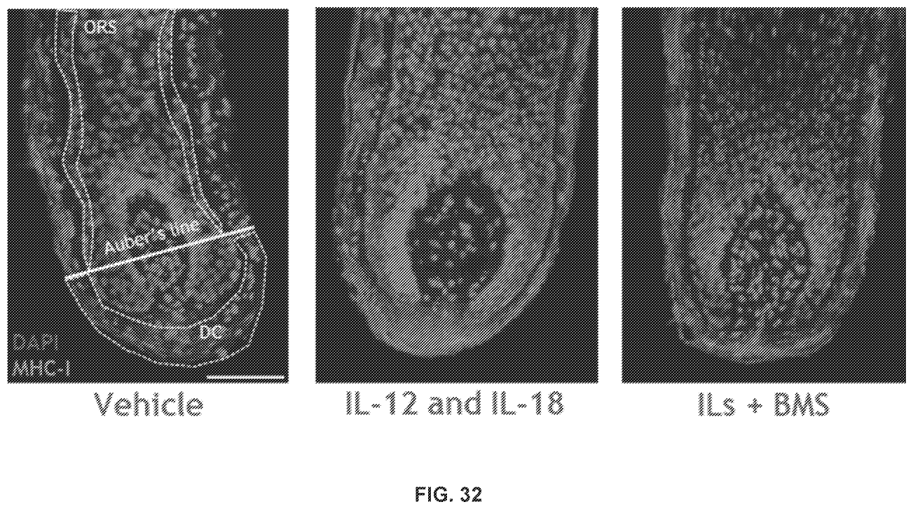

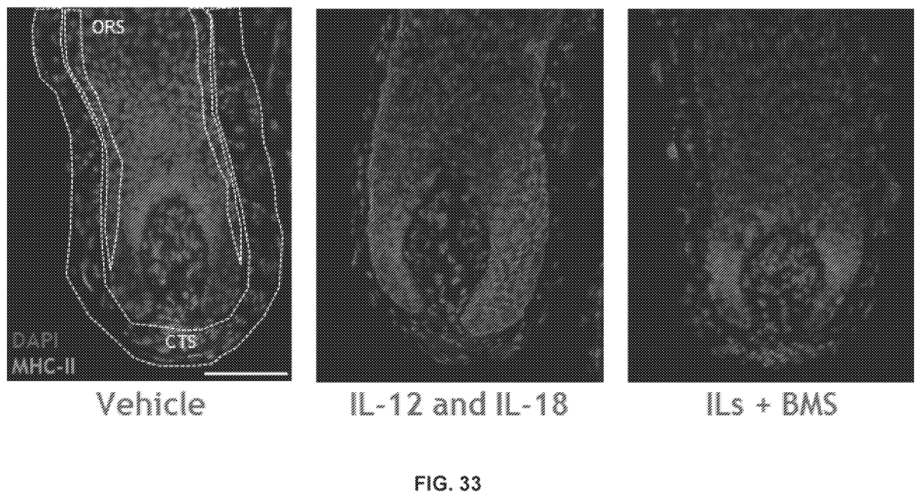

To assess whether TYK2 inhibition could restore hair follicle immune privilege, 21-31 hair follicles, obtained from 3-4 independent healthy donors, were cultured with vehicle, or with IL-12 (3 ng/mL) and IL-18 (20 ng/ml), for 5-6 days. On day 2 of culture, the compound of Formula (II) (300 nM) or tofacitinib (400 nM) was added until day 5-6. MHC class I expression and MHC class II expression were measured, as was done previously. See FIGS. 28-33. The graphs in FIGS. 28-31 show mean±SEM, with p-values from the Dunn's multiple comparison test (*p<0.05; **p<0.01; ***p<0.001; ****p<0.0001). FIGS. 32 and 33 show staining of MHC-I and MHC-II, respectively, in hair follicles (scale bar=100 μm). The compound of Formula (II) and tofacitinib each significantly reduced IL-12+IL-18-induced expression of MHC class I and of MHC class II. These results show that TYK2 inhibition can restore MHC class I and MHC class II levels to the levels observed in vehicle-treated hair follicles (which reflect the levels in healthy hair follicles). As discussed above, MHC class I and II are markers of immune privilege collapse.

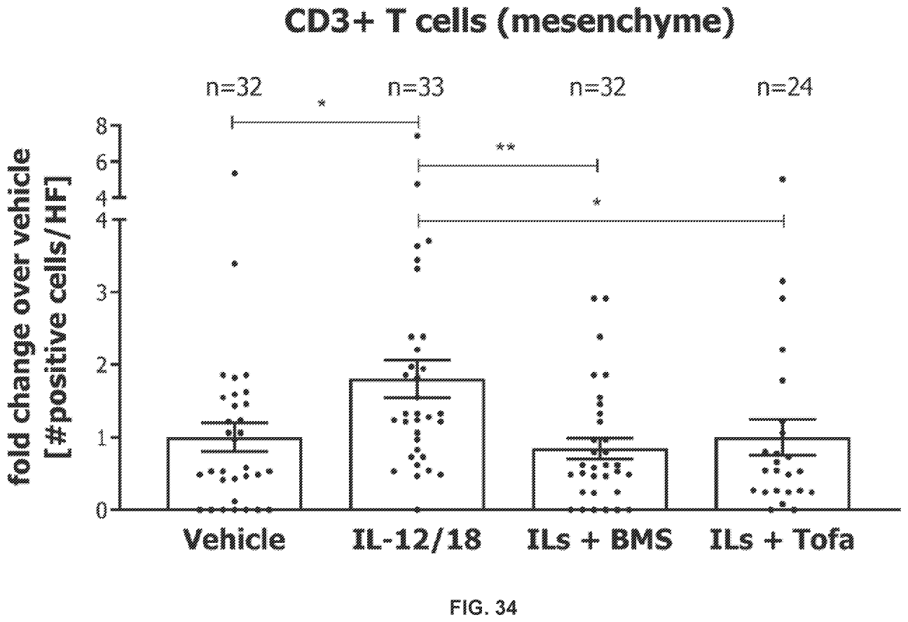

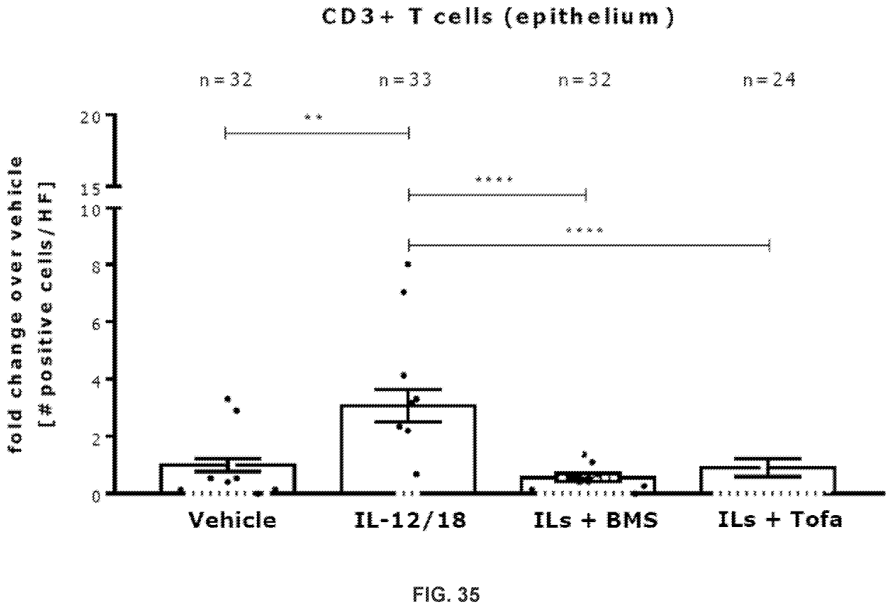

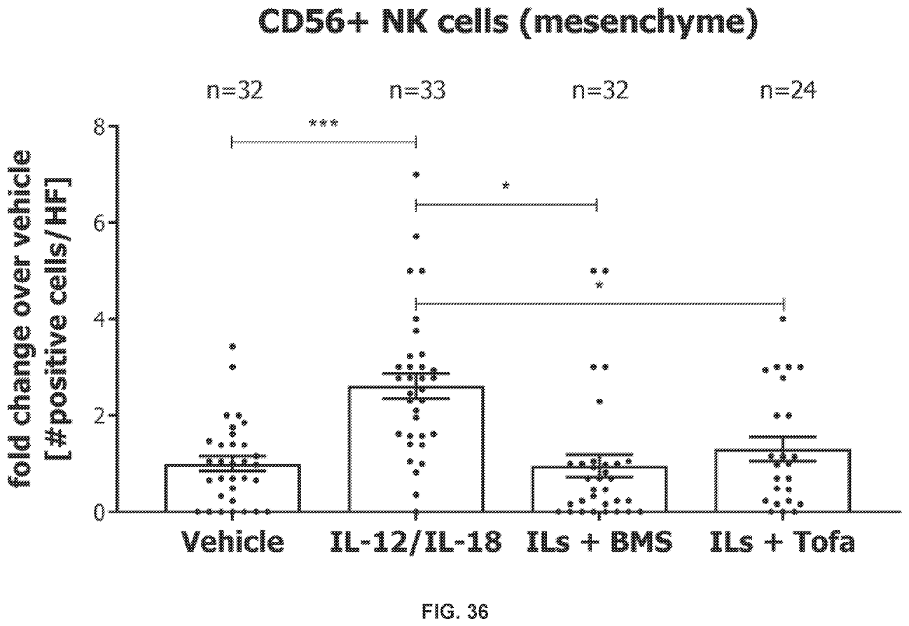

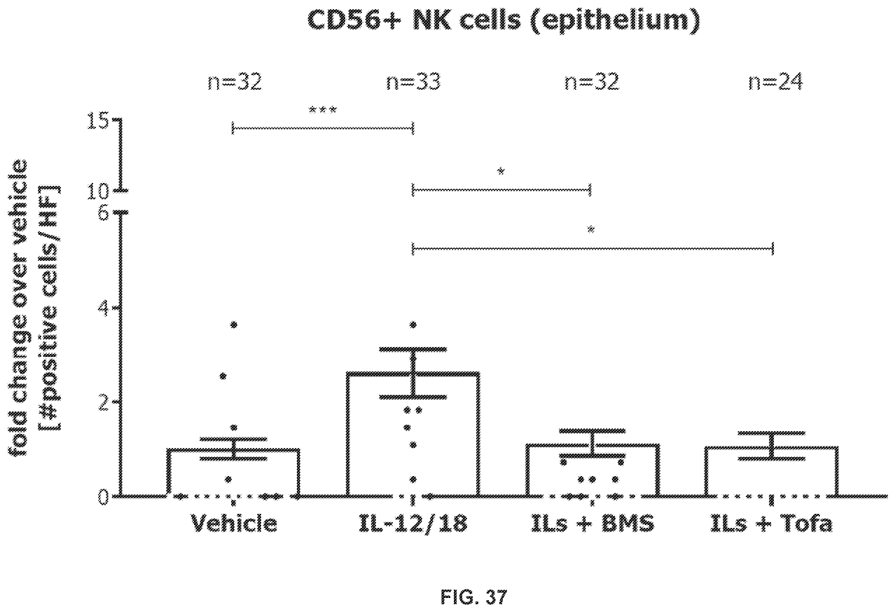

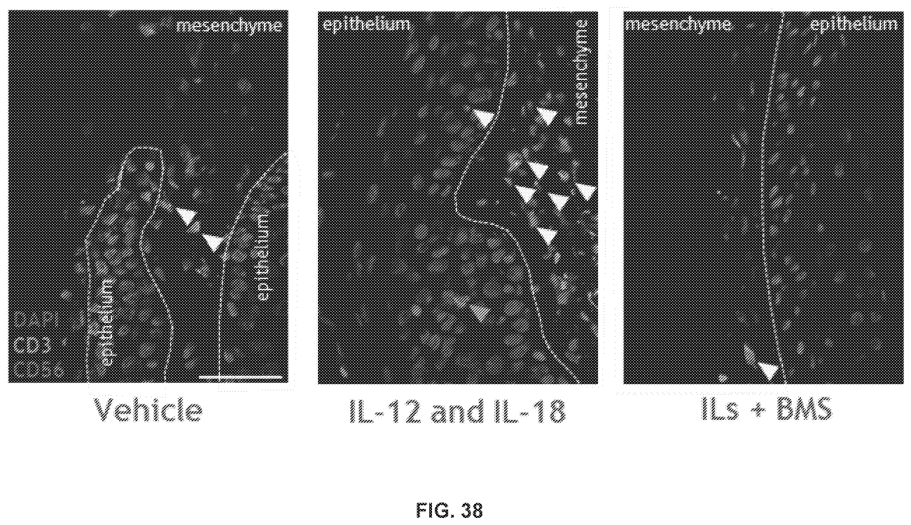

To assess whether TYK2 inhibition can block ongoing T cell and NK cell expansion, hair follicles were first incubated with vehicle, or with IL-12+IL-18, followed by the addition of the compound of Formula (II) or of tofacitinib. Specifically, 24-33 hair follicles, obtained from 3-4 independent healthy donors, were treated with vehicle, or with IL-12 (3 ng/ml) and IL-18 (20 ng/ml), for 5-6 days. On day 2 of culture, the compound of Formula (II) (300 nM) or tofacitinib (400 nM) was added. T cell expansion and NK cell expansion were assessed by immunostaining for CD3 (T cells) or CD56 (NK cells). The graphs in FIGS. 34-37 show mean±SEM, with p-values from the Dunn's multiple comparison test (*p<0.05; **p<0.01; ***p<0.001; ****p<0.0001). FIG. 38 provides images of staining for CD3 and CD56 in hair follicles (scale bar=50 μm).

Treatment the compound of Formula (II) rescued hair follicles from increases in T cells and in NK cells in the hair follicle epithelium and mesenchyme following IL-12+IL-18 stimulation. As shown in FIGS. 34-37, TYK2 inhibition with the compound of Formula (II) achieved T cell and NK cell numbers similar to the numbers observed in vehicle-treated hair follicles; the compound reversed the T cell and NK cell expansion induced by IL-12 and IL-18.

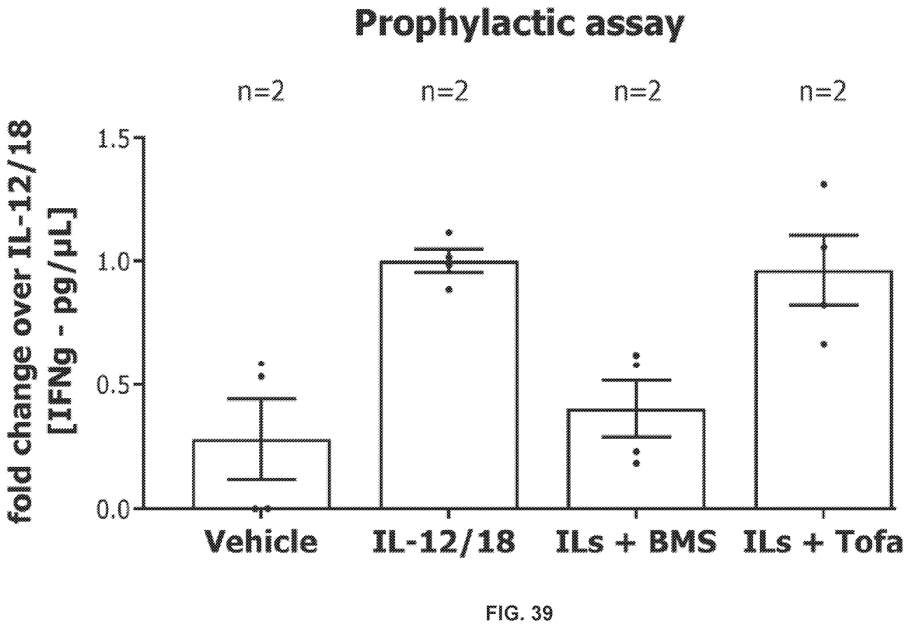

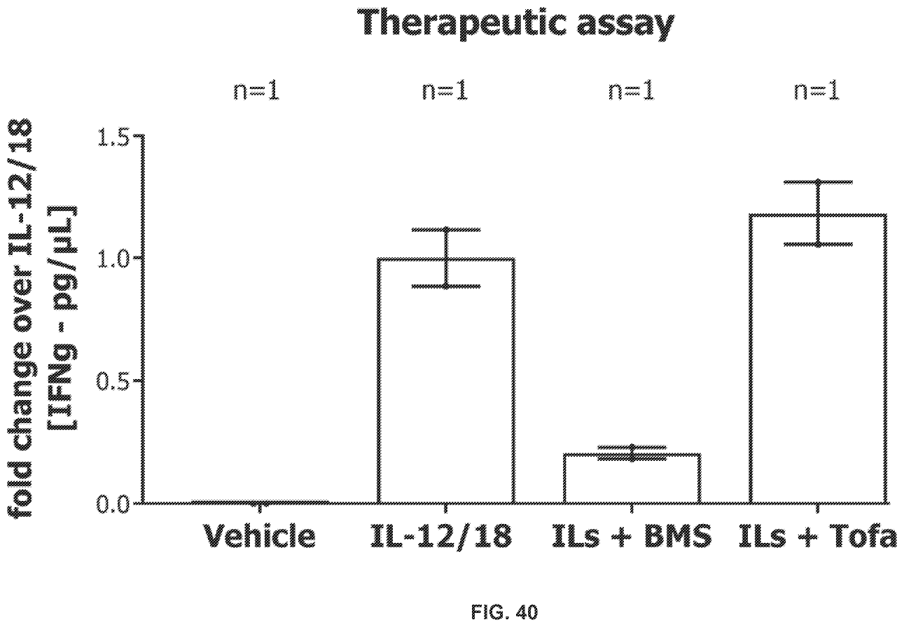

The effect of TYK2 inhibition on IFNγ production by hair follicles was also assessed. Specifically, two assays were performed to examine how TYK2 inhibition affects IFNγ secretion into the medium of ex vivo cultured hair follicles treated with IL-12+IL-18. In the prophylactic assay, hair follicles were pre-treated with the compound of Formula (II) or with tofacitinib, and then were cultured in the presence of IL-12+IL-18, after which IFNγ in the culture medium was measured. See FIG. 39. In the therapeutic assay, hair follicles were initially treated with IL-12±IL-18; then the compound of Formula (II) or tofacitinib was added and the culture continued, after which IFNγ in the culture medium was measured. See FIG. 40. The data in FIGS. 39 and 40 are based on the amount of IFNγ measured in pooled culture medium from n=8 hair follicles per group, obtained from 1-2 healthy donors. The graphs show mean±SEM from technical duplicates.

Local stimulation with IL-12+IL-18 leads to IFNγ release from hair follicles ex vivo, as shown in FIGS. 39 and 40. TYK2 inhibition can prevent IFNγ secretion into the medium, as well as restore the secretion to almost baseline levels, while such restoration was not observed with tofacitinib.

The above examples show that IL-12 is a key effector cytokine in promoting IFNγ secretion, immune cell expansion, and HF-IP collapse. Notably, tofacitinib treatment did not decrease IFNγ release into the culture medium. In contrast to tofacitinib, the compound of Formula (II) is a TYK2 inhibitor that directly and selectively targets TYK2 and the IL-12 receptor signaling pathway, thereby inhibiting IFNγ release.

### Example 4: IL-12 Receptor Expression in AA

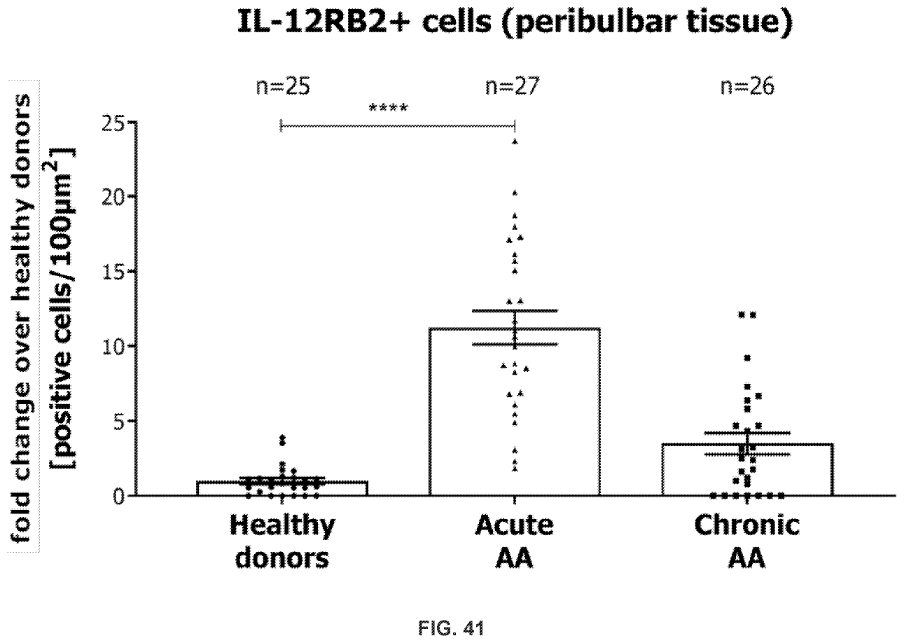

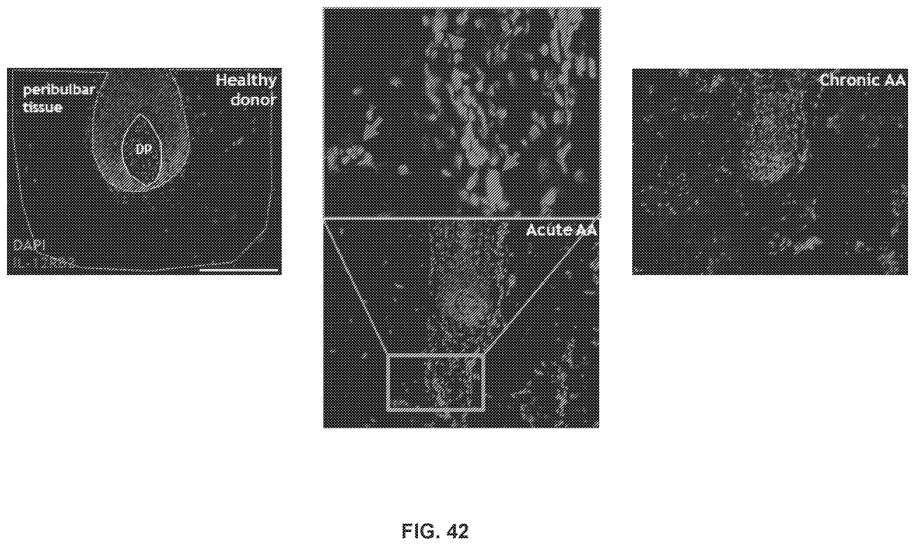

To confirm that TYK2 inhibition is an attractive new target for alopecia areata management, expression of the IL-12 receptor in freshly obtained lesional skin from acute and chronic AA patients was compared to IL-12 receptor expression in skin from healthy controls. IL-12RB2-expressing cells were evaluated in healthy donors, in acute AA patients, and in chronic AA patients using immunohistomorphometry. FIG. 41 provides data from 25-27 hair follicles per group, obtained from scalp biopsies of 3-4 independent donors (healthy donors, patients with acute AA, or patients with chronic AA, as indicated). For the immunostaining in FIG. 42, scale bar=200 μm.

As shown in FIG. 41, a higher number of IL-12RB2+ cells were observed around the bulb in skin from acute AA patients, compared to the number of IL-12RB2+ cells in skin from healthy individuals. This result further supports a role of IL-12 signaling in AA pathogenesis. IL-12RB2+ cells were also observed in chronic AA patients, suggesting that remaining resident immune cells retain the ability to respond to IL-12 stimulation.

While this invention has been particularly shown and described with reference to preferred embodiments thereof, it will be understood by those skilled in the art in light of the present disclosure that various changes in form and detail may be made therein without departing from the scope of the invention encompassed by the appended claims.

What is claimed is:

1. A method of treating a hair-loss disorder in a mammalian subject, the method comprising administering to the mammalian subject a TYK2 inhibitor.

2. The method according to claim 1, wherein the hair-loss disorder is alopecia areata.

3. The method according to claim 1, wherein the hair-loss disorder is alopecia totalis.

4. The method according to claim 1, wherein the hair-loss disorder is alopecia universalis.

5. The method according to any one of claims 1-4, wherein the TYK2 inhibitor is deucravacitinib.

6. The method according to any one of claims 1-4, wherein the TYK2 inhibitor is a pharmaceutically-acceptable salt of a compound having the structure of Formula (I):

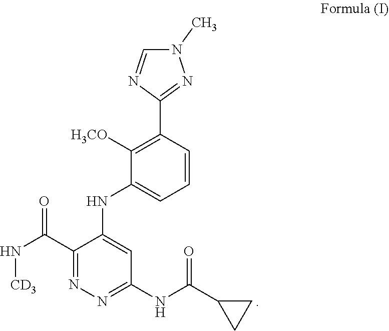 [SMILES: [2H]C([2H])([2H])NC(=O)c1nnc(NC(=O)C2CC2)cc1Nc1cccc(-c2ncn(C)n2)c1OC]

7. The method according to any one of claims 1-4, wherein the TYK2 inhibitor is a compound having the structure of Formula (II):

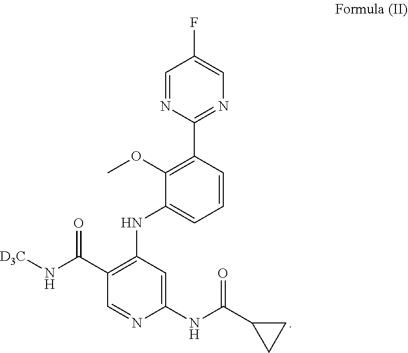 [SMILES: [2H]C([2H])([2H])NC(=O)c1cnc(NC(=O)C2CC2)cc1Nc1cccc(-c2ncc(F)cn2)c1OC]

8. The method according to any one of claims 1-4, wherein the TYK2 inhibitor is a pharmaceutically-acceptable salt of a compound having the structure of Formula (II):

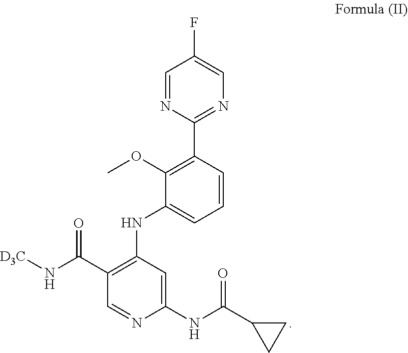 [SMILES: [2H]C([2H])([2H])NC(=O)c1cnc(NC(=O)C2CC2)cc1Nc1cccc(-c2ncc(F)cn2)c1OC]

9. The method according to any one of claims 1-8, wherein the TYK2 inhibitor is administered orally.

10. The method according to any one of claims 1-8, wherein the TYK2 inhibitor is administered topically.

11. A method of preventing hair loss in a mammalian subject, the method comprising administering to the mammalian subject a TYK2 inhibitor, wherein the mammalian subject has previously suffered from alopecia areata.

12. A method of preventing hair loss in a mammalian subject, the method comprising administering to the mammalian subject a TYK2 inhibitor, wherein the mammalian subject has previously suffered from alopecia totalis.

13. A method of preventing hair loss in a mammalian subject, the method comprising administering to the mammalian subject a TYK2 inhibitor, wherein the mammalian subject has previously suffered from alopecia universalis.

14. The method according to any one of claims 11-13, wherein the TYK2 inhibitor is a compound having the structure of Formula (I) or a pharmaceutically-acceptable salt thereof:

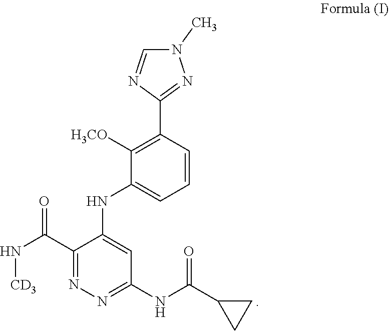 [SMILES: [2H]C([2H])([2H])NC(=O)c1nnc(NC(=O)C2CC2)cc1Nc1cccc(-c2ncn(C)n2)c1OC]

15. The method according to any one of claims 11-13, wherein the TYK2 inhibitor is a compound having the structure of Formula (II) or a pharmaceutically-acceptable salt thereof:

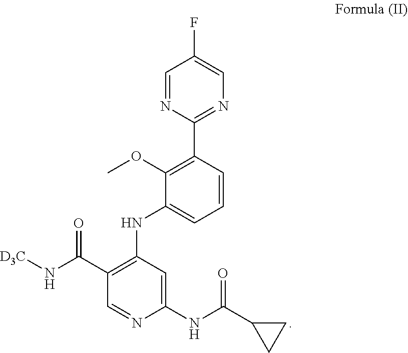 [SMILES: [2H]C([2H])([2H])NC(=O)c1cnc(NC(=O)C2CC2)cc1Nc1cccc(-c2ncc(F)cn2)c1OC]

16. The method according to any one of claims 11-15, wherein the TYK2 inhibitor is administered orally.

17. The method according to any one of claims 11-15, wherein the TYK2 inhibitor is administered topically.

18. The method according to any one of claims 1-10, wherein said administering the TYK2 inhibitor to the mammalian subject comprises administering the TYK2 inhibitor at least twice per week.

19. The method according to claim 18, wherein the TYK2 inhibitor is administered to the mammalian subject once daily.

20. The method according to any one of claims 18-19, wherein the method further comprises promoting regrowth of hair in the mammalian subject.

21. A method of regrowing hair in a human subject suffering from alopecia areata, the method comprising administering to the human subject a TYK2 inhibitor.

22. The method according to claim 21, wherein the TYK2 inhibitor is a compound having the structure of Formula (I) or a pharmaceutically-acceptable salt thereof:

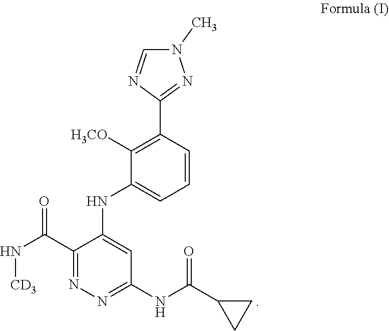 [SMILES: [2H]C([2H])([2H])NC(=O)c1nnc(NC(=O)C2CC2)cc1Nc1cccc(-c2ncn(C)n2)c1OC]

23. The method according to claim 21, wherein the TYK2 inhibitor is a compound having the structure of Formula (II) or a pharmaceutically-acceptable salt thereof: 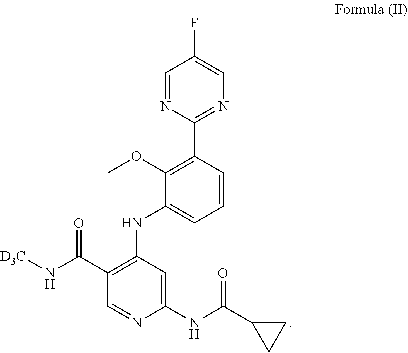 [SMILES: [2H]C([2H])([2H])NC(=O)c1cnc(NC(=O)C2CC2)cc1Nc1cccc(-c2ncc(F)cn2)c1OC]
